(19) United States
(12) Patent Application Publication
(10) Pub. No.: US 2013/0183266 A1
(43) Pub. Date: Jul. 18, 2013

(54) COMPOUNDS AND METHODS FOR THE TREATMENT OR PREVENTION OF FLAVIVIRUS INFECTIONS

(71) Applicant: Vertex Pharmaceuticals Incorporated, Cambridge, MA (US)

(72) Inventors: Jeremy Green, Waltham, MA (US); Dean M. Wilson, Bedford, MA (US); Laval Chan Chun Kong, Kirkland (CA); Sanjoy Kumar Das, Pierrefonds (CA); Real Denis, Laval (CA)

(73) Assignee: Vertex Pharmaceuticals Incorporated, Cambridge, MA (US)

(21) Appl. No.: 13/724,068

(22) Filed: Dec. 21, 2012

Related U.S. Application Data
(63) Continuation of application No. PCT/US2011/042141, filed on Jun. 28, 2011.
(60) Provisional application No. 61/359,156, filed on Jun. 28, 2010.

(51) Int. Cl. A61K 31/381; A61K 31/7056; A61K 38/21; C07D 333/36; A61K 45/06

(52) U.S. Cl. CPC A61K 31/381; C07D 333/36; A61K 45/06; A61K 38/21; A61K 31/7056
USPC 424/85.4; 549/69; 514/447; 435/184; 514/43

(57) ABSTRACT

A compound is represented by Structural Formula (I):

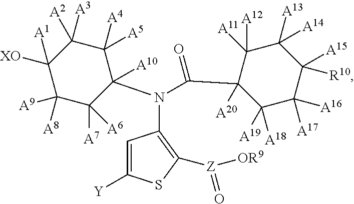

or a pharmaceutically acceptable salt thereof, wherein the variables of Structural Formula (I) are as described in the specification and the claims. A pharmaceutical composition comprises a compound represented by Structural Formula (I) or a pharmaceutically acceptable salt thereof, and a pharmaceutically acceptable carrier of excipient. A biological probe comprises a compound represented by Structural Formula (I) or a pharmaceutically acceptable salt thereof. A method of treating a HCV infection in a subject comprises administering to the subject a therapeutically effective amount of a compound represented by Structural Formula (I) or a pharmaceutically acceptable salt thereof. A method of inhibiting or reducing the activity of HCV polymerase in a subject or in a biological in vitro sample comprises administering to the subject or to the sample a therapeutically effective amount of a compound represented by Structural Formula (I) or a pharmaceutically acceptable salt thereof.

### RELATED APPLICATIONS

This application is a continuation of PCT Application Number PCT/US2011/042141, filed Jun. 28, 2011, which claims priority to U.S. Provisional Application Ser. No. 61/359,156 filed on Jun. 28, 2010. The entire teachings of this application are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flavivirus infections.

### SUMMARY OF THE INVENTION

The present invention generally relates to compounds useful for treating or preventing Flavivirus infections, such as HCV infections, and/or as analytical tools or probes in biological assays.

In one embodiment, the invention is directed to a compound represented by Structural Formula (I):

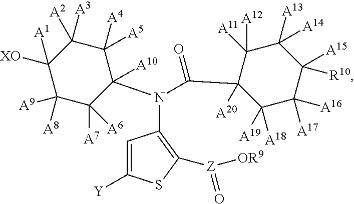

or a pharmaceutically acceptable salt thereof, wherein:

X is —H, [—C(O)C(R⁴R⁵)N(R)-]ₙ—C(O)C(R⁶R⁷R⁸), —P(O)(OR³)₂, or —C(O)R².

Y is —C≡CR¹,

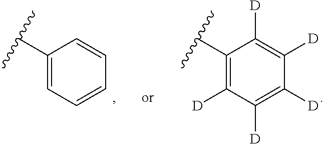 [SMILES: *c1ccccc1 ; *c1c([2H])c([2H])c([2H])c([2H])c1[2H]]

Z is C or C¹⁴.

Each of A¹-A²⁰ independently is —H or -D (deuterium).

R¹ is —H or a C₁₋₆ alkyl, C₃₋₁₀ carbocyclic, 4-10 membered heterocyclic, C₆₋₁₀ aryl, or 5-10 membered heteroaryl group, wherein said alkyl group is optionally substituted with one or more instances of J^{1A}, and wherein each of said carbocyclic and heterocyclic groups is optionally and independently substituted with one or more instances of J^{1B}, and wherein each of said aryl and heteroaryl groups is optionally and independently substituted with one or more instances of J^{1C}.

R² is a C₃₋₁₀carbocyclic, 4-10 membered heterocyclic, C₆₋₁₀ aryl, or 5-10 membered heteroaryl group, wherein each of said carbocyclic and heterocyclic groups is independently and optionally substituted with one or more instances of J^{E}, and each of said aryl and heteroaryl groups is independently and optionally substituted with one or more instances of J^{F}.

R³ is —H, a C₁₋₆ aliphatic, C₃₋₁₀ carbocyclic, 4-10 membered heterocyclic, C₆₋₁₀ aryl, or 5-10 membered heteroaryl group, wherein said aliphatic group is optionally substituted with one or more instances of J^{D}, each of said carbocyclic and heterocyclic groups is independently and optionally substituted with one or more instances of J^{E}, and each of said aryl and heteroaryl groups is independently and optionally substituted with one or more instances of J^{F}.

Each of R⁴, R⁵, R⁶, and R⁷ independently is —H; or C₁₋₆ alkyl optionally substituted with one or more substitutents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O)N(C₁₋₆ alkyl)₂, —NHC(O)(C₁₋₆ alkyl), phenyl, hydroxyphenyl, imidazole, and indole.

R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}.

R⁹ is: i) —H; ii) a C₁₋₆ aliphatic group optionally substituted with one or more one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}.

R¹⁶ is —CH₃, —CH₂D, —CHD₂, or —CD₃.

Each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E}.

Each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E}.

Each of J^{1C} and J^{9C} independently is Q or a C₁₋₆ aliphatic group optionally substituted with one or more instances of Q; or two J^{1C} and two J^{9C}, respectively, together with the atoms to which they are attached, optionally and independently form a 3-8-membered non-aromatic ring that is optionally substituted with one or more instances of J^{E}.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(ORa)ORa, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{E}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}.

Each R^{a}, R^{b}, and R^{c} independently is: i) —H; ii) a C₁₋₆ aliphatic group optionally substituted with one or more substituents independently selected from the group consisting of halogen, oxo, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or

R^{a}, together with R and the nitrogen atom to which it is attached, optionally forms a 4-8 membered heterocycle optionally substituted with one or more instances of J^{E}; or

R^{b} and R^{e}, together with the nitrogen atom to which they are attached, optionally forms a 4-8 membered heterocycle optionally substituted with one or more instances of J^{E}.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

Each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

Provided that when Y is —C≡CR¹ or

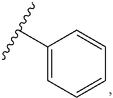 [SMILES: *c1ccccc1]

Z is C, and R¹⁰ is —CH₃, then at least one of A¹-A²⁰ is -D.

In another embodiment, the invention is directed to a pharmaceutical composition comprising a compound of the invention described herein (e.g., a compound selected from the compounds described in the claims and FIG. 1, such as a compound represented by any one of Structural Formulae (I)-(XVIII)) or a pharmaceutically acceptable salt thereof) and a pharmaceutically acceptable carrier or excipient.

In yet another embodiment, the invention provides methods of treating a HCV infection in a subject, comprising administering to the subject a therapeutically effective amount of a compound of the invention described herein (e.g., a compound selected from the compounds described in the claims and FIG. 1, such as a compound represented by any one of Structural Formulae (I)-(XVIII) or a pharmaceutically acceptable salt thereof).

In yet another embodiment, the invention is directed to a method of inhibiting or reducing the activity of HCV polymerase in a subject, comprising administering to the subject a therapeutically effective amount of a compound of the invention described herein (e.g., a compound selected from the compounds described in the claims and FIG. 1, such as a compound represented by any one of Structural Formulae (I)-(XVIII) or a pharmaceutically acceptable salt thereof).

In yet another embodiment, the invention is directed to a method of inhibiting or reducing the activity of HCV polymerase in a biological in vitro sample, comprising administering to the sample an effective amount of a compound of the invention described herein (e.g., a compound selected from the compounds described in the claims and FIG. 1, such as a compound represented by any one of Structural Formulae (I)-(XVIII) or a pharmaceutically acceptable salt thereof).

The present invention also provides use of the compounds of the invention described herein (e.g., the compounds described in the claims and FIG. 1, such as the compounds represented by Structural Formulae (I)-(XVIII) or pharmaceutically acceptable salts thereof), for the manufacture of the medicament for treating a HCV infection in a subject, or for inhibiting or reducing the activity of HCV polymerase in a subject.

Also provided herein is use of the compounds of the invention described herein (e.g., the compounds described in the claims and FIG. 1, such as the compounds represented by Structural Formulae (I)-(XVIII) or pharmaceutically acceptable salts thereof) for treating a HCV infection in a subject, or for inhibiting or reducing the activity of HCV polymerase in a subject.

### DESCRIPTION OF THE DRAWINGS

FIG. 1 shows a table depicting certain compounds of the invention.

### DETAILED DESCRIPTION OF THE INVENTION

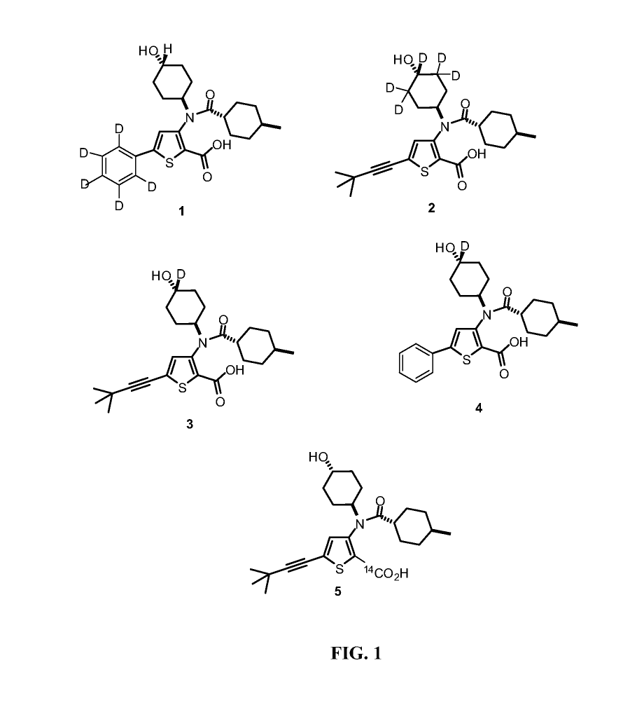

The compounds of the invention are as described in the claims. In some embodiments, the compounds of the invention are represented by any one of Structural Formulae (I)-(XVIII) or pharmaceutically acceptable salts thereof, wherein the variables are each and independently as described in any one of the claims. In some embodiments, the compounds of the invention are represented by any chemical formulae depicted in FIG. 1 or pharmaceutically acceptable salts thereof. In some embodiments, the compounds of the invention are presented by any one of Structural Formulae (I)-(XVIII) or pharmaceutically acceptable salts thereof, wherein the variables are each and independently as depicted in the chemical formulae in FIG. 1.

In one embodiment, the compounds of the invention are represented by Structural

Formula (I) or (II):

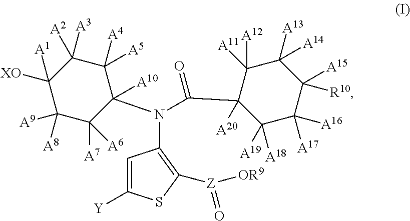

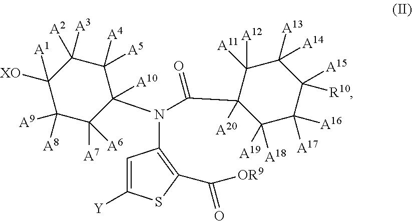

or a pharmaceutically acceptable salt thereof, wherein the values of the variables of Structural Formula (I) are as follows:

In the first set of the values of the variables of Structural Formulae (I) and (II):

X is —H, [—C(O)C(R⁴R⁵)N(R)-]ₙ—C(O)C(R⁶R⁷R⁸), —P(O)(OR³)₂, or —C(O)R². In one aspect, X is —H, [—C(O)C(R⁴R⁵)N(R)-]ₙ—C(O)C(R⁶R⁷R⁸), or —P(O)(OR³)₂. In another aspect, X is —H or [—C(O)C(R⁴R⁵)N(R)—]ₙ—C(O)C(R⁶R⁷R⁸).

Y is —C≡CR¹,

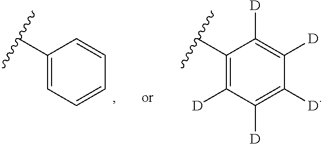 [SMILES: *c1ccccc1 ; *c1c([2H])c([2H])c([2H])c([2H])c1[2H]]

Z is ¹²C or C¹⁴. Specifically, Z is ¹²C.

Each of A¹-A²⁰ independently is —H or -D. In one aspect, at least one of A¹-A¹⁰ is -D, and each of A¹¹-A²⁰ is —H. In another aspect, at least one of A¹, A², A³, A⁸ and A⁹ is A⁹-D. In yet another aspect, A¹ is -D; and A², A³, A⁸, and Aare —H. In yet another aspect, A¹, A², A³, A⁸, and A⁹ are -D. In yet another aspect, at least one of A¹¹-A²⁰ is -D and each of A¹-A¹⁰ is H. In yet another aspect, at least one of A¹¹-A²⁰ is D, D and at least one of A¹-A¹⁰ is —H.

R¹ is: i) —H; ii) a C₁₋₆ aliphatic group optionally substituted with one or more one or more instances of J^{1A}; iii) a C₃₋₁₀ carbocycle or 4-10 membered heterocycle, each of which is optionally and independently substituted with one or more instances of J^{1B}; or iv) a C₆₋₁₀ aryl or 5-10 membered heteroaryl group, each of which is optionally and independently substituted with one or more instances of J^{1C}. In one aspect, R¹ is an optionally substituted C₁₋₆ alkyl or optionally substituted C₃₋₈ carbocyclic group. In another aspect, R¹ is an optionally substituted C₁₋₆ alkyl or optionally substituted C₃₋₁₀ cycloalkyl group. In yet another aspect, R¹ is an optionally substituted C₁₋₆ alkyl or C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In yet another aspect, R¹ is C₁₋₆ alkyl or C₃₋₈ cycloalkyl, each of which optionally and independently substituted with one or more substituents selected from the group consisting of halogen, —CN, —OH, —O(C₁₋₆alkyl), and —O(C₁₋₆ haloalkyl). In yet another aspect, R¹ is C₁₋₆ alkyl or C₃₋₈ cycloalkyl. In yet another aspect, R¹ is C₁₋₆ alkyl. In yet another aspect, R¹ is t-butyl or isopropyl.

R² is: i) a C₃₋₁₀ carbocyclic or 4-10 membered heterocyclic group, each of which is independently and optionally substituted with one or more instances of J^{E}; or ii) a C₆₋₁₀ aryl or 5-10 membered heteroaryl group, each of which is independently and optionally substituted with one or more instances of J^{F}.

R³ is i) —H, ii) a C₁₋₆ aliphatic group optionally substituted with one or more instances of J^{D}, iii) a C₃₋₁₀ carbocyclic or 4-10 membered heterocyclic group, each of which is independently and optionally substituted with one or more instances of J^{E}; or iv) a C₆₋₁₀ aryl or 5-10 membered heteroaryl group, each of which is independently and optionally substituted with one or more instances of J^{F}. In one aspect, each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. In another aspect, each R³ is —H or an optionally substituted C₁₋₆ aliphatic group. In yet another aspect, each R³ independently is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In yet another aspect, each R³ independently is —H or C₁₋₆ alkyl.

Each of R⁴, R⁵, R⁶, and R⁷ independently is —H; or C₁₋₆ alkyl optionally substituted with one or more substituents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O)N(C₁₋₆ alkyl)₂, —NHC(O)(C₁₋₆ alkyl), phenyl, hydroxyphenyl, imidazole, and indole. In one aspect, each of R⁴, R⁵, R⁶, and R⁷ independently is —H; or C₁₋₄ alkyl optionally substituted with one or more substituents selected from the group consisting of —OH, —NH₂, —NH—C(═NH)—NH₂, —CO₂H, —C(O)NH₂, phenyl, hydroxyphenyl, imidazole, and indole. In another aspect, each of R⁴, R⁵, R⁶ and R⁷ independently —H, or C₁₋₄ alkyl optionally substituted with one or more substituents selected from the group consisting of —OH, —NH₂, —NH—C(═NH)—NH₂, —CO₂H, —C(O)NH₂, phenyl, and hydroxyphenyl. In yet another aspect, each of R⁴, R⁵, R⁶ and R⁷ independently is —H or C₁₋₆ alkyl optionally substituted with one or more substituents selected from the group consisting of —OH, —NH₂, —NH—C(═NH)—NH₂, —CO₂H, and —C(O)NH₂. In yet another aspect, each of R⁴, R⁵, R⁶, and R⁷ independently is —H, C₁₋₄ alkyl, —CH₂CO₂H, —CH₂—CH₂—CO₂H, —CH₂—(CH₂)₃—NH₂, —CH₂—(CH₂)₂—NH—C(═NH)—NH₂, —CH₂(Phenyl), —CH₂(p-hydroxyphenyl), —CH₂OH, —CH(OH)CH₃, —CH₂C(O)NH₂, or —CH₂CH₂C(O)NH₂. In yet another aspect, R⁴ and R⁶ are each independently —H or C₁₋₆ alkyl; and R⁵ and R⁷ are each independently —H or optionally substituted C₁₋₆ alkyl. In yet another aspect, each of R⁴, R⁵, R⁶, and R⁷ independently is —H, or C₁₋₄ alkyl.

R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}. In one aspect, R⁸ independently is —H, halogen, cyano, —OR^{b}, —NR^{b}R^{c}, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. In another aspect, R⁸ is —NR^{b}R^{c}.

R⁹ is: i) —H; ii) a C₁₋₆ aliphatic group optionally substituted with one or more one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}. In one aspect, R⁹ is —H, or an optionally substituted C₁₋₆ aliphatic or optionally substituted carbocyclic group. In another aspect, R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —OC(O)O(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), phenyl, and 5-6 membered heterocycle optionally substituted with one or more substituents selected from the group consisting of oxo and C₁₋₆ alkyl. In yet another aspect, R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In yet another aspect, R⁹ is —H.

R¹⁰ is —CH₃, —CH₂D, —CHD₂, or —CD₃. In one aspect, R¹⁰ is —CH₃.

Each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E}. In one aspect, each of J^{1A} and J^{9A} independently is halogen, oxo, —CN, —OR^{a}, —NRR^{a}, —OCOR^{a}, —OCOOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), optionally substituted phenyl, or optionally substituted 5-6 membered heterocyclyl. In another aspect, each of J^{1A} and J^{9A} independently is halogen, oxo, —CN, —OR^{a}, —N^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl.

Each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E}. In one aspect, each of J^{1B} and J^{9B} independently is halogen, oxo, —CN, —OR^{a}, N^{a}—RR, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Each of J^{1C} and J⁹ independently is Q or a C₁₋₆ aliphatic group optionally substituted with one or more instances of Q; or two J^{1C} and two J^{9C}, respectively, togetherC with the atoms to which they are attached, optionally and independently form a 3-8-membered non-aromatic ring that is optionally substituted with one or more instances of J^{E}. In one aspect, each of J^{1C} and J^{9C} independently is halogen, oxo, —CN, —OR^{a}, —N^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —N^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Each Q independently is selected from the group consisting of halogen, cyano, nitro, —OR^{a}, —SRa, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(ORa)ORa, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. In one aspect, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. In another aspect, each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}; —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl.

Each R^{a}, R^{b}, and R^{c} independently is: i) —H; ii) a C₁₋₆ aliphatic group optionally substituted with one or more substituents independently selected from the group consisting of halogen, oxo, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or R^{a}, together with R and the nitrogen atom to which it is attached, optionally forms a 4-8 membered heterocycle optionally substituted with one or more instances of J^{E}; or R^{b} and R^{e}, together with the nitrogen atom to which they are attached, optionally forms a 4-8 membered heterocycle optionally substituted with one or more instances of J^{E}. In one aspect, R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring.

In another aspect, each of R^{b} and R^{c} independently is —H or an optionally substituted C₁-C₆ aliphatic group, or optionally, together with the nitrogen atom to which they are attached, form an optionally substituted 4-8 membered heterocyclic ring. In yet another aspect, each of R^{a}, R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(Cl_{—}6 alkyl), —O(C₁₋₆alkyl), —O(C₁₋₆ haloalkyl), C₃₋₇ cycloalkyl, C₃₋₇ cyclo(haloalkyl), and phenyl; or R^{b} and R^{c}, together with the nitrogen atom to which they are attached, form a 5-7 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆alkyl), and —O(C₁₋₆haloalkyl). In yet another aspect, each of R^{b} and R^{c} independently is —H or C₁₋₄ alkyl.

Each R is independently —H or a C₁₋₆ aliphatic group optionally substituted with one or more instances of J^{D}, or optionally R^{a}, together with R and the nitrogen atom to which it is attached, forms an optionally substituted 5-8 membered heterocyclic ring.

Each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

Each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

Provided that when Y is —C≡CR¹ or

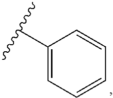 [SMILES: *c1ccccc1]

Z is C, and R¹⁰ is —CH₃, then at least one of A¹-A²⁰ is -D.

A second set of values of the variables of Formulae (I) and (II) is as set forth below:

X is —H, [—C(O)C(R⁴R⁵)N(R)-]ₙ—C(O)C(R⁶R⁷R⁸), or —P(O)(OR³)₂.

Values of the other variables of Structural Formulae (I) and (II) are each and independently as described above in the first set of values of the variables of Structural Formulae (I) and (II).

A third set of values of the variables of Structural Formulae (I) and (II) is as set forth below:

Each Q independently is selected from the group consisting of halogen; cyano; nitro; —OR^{a}; —SR^{a}; —S(O)R^{a}; —SO₂R^{a}; —N^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}; —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl.

Values of the other variables of Structural Formulae (I) and (II) are each and independently as described above in the first set of values of the variables of Structural Formulae (I) and (II)

A fourth set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

Each Q independently is selected from the group consisting of halogen; cyano; nitro; —OR^{a}; —SR^{a}; —S(O)R^{a}; —SO₂R^{a}; —N^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}; —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl.

Values of the other variables of Structural Formulae (I) and (II) are each and independently as described above in the first set of values of the variables of Structural Formula (I).

A fourth set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

Each Q independently is selected from the group consisting of halogen; cyano; nitro; —OR^{a}; —SRa; —S(O)R^{a}; —SO₂R^{a}; —NRR^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}; —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Suitable substituents are as described above in the first set of values of the variables of Structural Formulae (I) and (II).

Values of the other variables of Structural Formulae (I) and (II) are each and independently as described above in the first set of values of the variables of Structural Formulae (I) and (II).

A fifth set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring. Suitable substituents are as described above in the first set of values of the variables of Structural Formulae (I) and (II).

Values of the other variables of Structural Formulae (I) and (II) are each and independently as described above in the first set of values of the variables of Structural Formulae (I) and (II).

A sixth set of values of the variables of Structural Formulae (I) and (II) is as set forth below:

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally Ra, together with R and the nitrogen atom to which it is attached, forms an optionally substituted 5-8 membered heterocyclic ring. Suitable substituents are as described above in the first set of values of the variables of Structural Formulae (I) and (II).

Each Q independently is selected from the group consisting of halogen; cyano; nitroc; —OR^{a}; —SRa; —S(O)Ra; —SO₂Ra; —NRR^{a}; —C(O)Ra; —C(O)ORa; —OC(O)Ra; —NRC(O)R^{a}; —C(O)NRR^{a}; —NRC(O)NRR^{a}; —NRC(O)OR^{a}; —NRC(═NR)NRR^{a}; —OCONRR^{a}; —C(O)NRC(O)ORa; —C(═NR)Ra; —C(═NOR)Ra; —SO₂NRR^{a}; —NRSO₂Ra; —NRSO₂NRR^{a}; —OP(O)(OR^{a})OR^{a}; option ally substituted C₃₋₈ carbocyclic; 4-8 membered, optionally substituted heterocyclyl; optionally substituted phenyl; and optionally substituted, 5-6 membered heteroaryl. Suitable substituents are as described above in the first set of values of the variables of Structural Formulae (I) and (II).

Values of the other variables of Structural Formulae (I) and (II) are each and independently as described above in the first set of values of the variables of Structural Formulae (I) and (II).

A seventh set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

R¹ is an optionally substituted C₁₋₆ alkyl or optionally substituted C₃₋₈ carbocyclic group.

Values of the other variables of Structural Formulae (I) and (II) are each and independently as described above in the first set of values of the variables of Structural Formulae (I) and (II).

An eighth set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

Each Q independently is as described above in the third set of values of the variables of Structural Formulae (I) and (II).

R¹ is an optionally substituted C₁₋₆ alkyl or optionally substituted C₃₋₈ carbocyclic group.

Values of the other variables of Structural Formulae (I) and (II) are each and independently as described above in the first set of values of the variables of Structural Formulae (I) and (II).

A ninth set of values of the variables of Structural Formulae (I) and (II) is as set forth below:

X is —H, [—C(O)C(R⁴R⁵)N(R)-]ₙ—C(O)C(R⁶R⁷R⁸), or —P(O)(OR³)₂.

Each Q independently is as described above in the third set of values of the variables of Structural Formulae (I) and (II).

R¹ is an optionally substituted C₁₋₆ alkyl or optionally substituted C₃₋₈ carbocyclic group.

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring. Suitable substituents are as described above in the first set of values of the variables of Structural Formulae (I) and (II).

Values of the other variables of Structural Formulae (I) and (II) are each and independently as described above in the first set of values of the variables of Structural Formulae (I) and (II).

A tenth set of values of the variables of Structural Formulae (I) and (II) is as set forth below:

Each of X, Q, R¹, and R^{a} is independently as described above in any one of the first through ninth sets of values of the variables of Structural Formulae (I) and (II).

Each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl.

Each of R⁴, R⁵, R⁶, and R⁷ independently is —H; or C₁₋₄ alkyl optionally substituted with one or more substituents selected from the group consisting of —OH, —NH₂, —NH—C(═NH)—NH₂, —CO₂H, —C(O)NH₂, phenyl, hydroxyphenyl, imidazole, and indole.

R⁸ independently is —H, halogen, cyano, —OR^{b}, —NR^{b}R^{c}, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl.

Values of the other variables of Structural Formulae (I) and (II) are each and independently as described above in the first set of values of the variables of Structural Formulae (I) and (II).

An eleventh set of values of the variables of Structural Formulae (I) and (II) is as set forth below:

Each of X, Q, R¹, R³, R⁴, R⁵, R⁶, R⁷, and R^{a} is independently as described above in any one of the first through tenth sets of values of the variables of Structural Formulae (I) and (II).

R⁸ is —NR^{b}R^{c}.

Values of the other variables of Structural Formulae (I) and (II) are each and independently as described above in the first set of values of the variables of Structural Formulae (I) and (II).

A twelfth set of values of the variables of Structural Formulae (I) and (II) is as set forth below:

Each of X, Q, R¹, R³, R⁴, R⁵, R⁶, R⁷, R⁸, and R^{a} is independently as described above in any one of the first through eleventh sets of values of the variables of Structural Formulae (I) and (II).

R⁹ is —H, or an optionally substituted C₁₋₆ aliphatic or optionally substituted carbocyclic group.

Values of the other variables of Structural Formulae (I) and (II) are each and independently as described above in the first set of values of the variables of Structural Formulae (I) and (II).

A thirteenth set of values of the variables of Structural Formulae (I) and (II) is as set forth below:

Each of X, Q, R¹, R³, R⁴, R⁵, R⁶, R⁷, R⁸, R⁹, and R^{a} is independently as described above in any one of the first through twelfth sets of values of the variables of Structural Formulae (I) and (II).

Each of J^{1A} and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), optionally substituted phenyl, or optionally substituted, 5-6 membered heterocyclyl. Specifically, each of J^{1A} and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl. Specifically, each J^{1A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl; and each J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), optionally substituted phenyl, or optionally substituted phenyl.

Each of J^{1B} and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Each of J^{1C} and J^{9C} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCORa, —CORa, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Values of the other variables of Structural Formulae (I) and (II) are each and independently as described above in the first set of values of the variables of Structural Formulae (I) and (II).

A fourteenth set of values of the variables of Structural Formulae (I) and (II) is as set forth below:

Each of X, Q, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is —C≡CR¹; and

R¹ is an optionally substituted C₁₋₆ alkyl or optionally substituted C₃₋₁₀ cycloalkyl group.

Values of the other variables of Structural Formulae (I) and (II) are each and independently as described above in the first set of values of the variables of Structural Formulae (I) and (II).

A fifteenth set of values of the variables of Structural Formulae (I) and (II) is as set forth below:

Each of X, Q, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

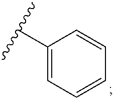 [SMILES: *c1ccccc1]

and

R¹ is an optionally substituted C₁₋₆ alkyl or optionally substituted C₃₋₁₀ cycloalkyl group.

Values of the other variables of Structural Formulae (I) and (II) are each and independently as described above in the first set of values of the variables of Structural Formulae (I) and (II).

A sixteenth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

At least one of A¹-A¹⁰ is D, u and each of A¹¹-A²⁰ is —H.

Values of the other variables of Structural Formulae (I) and (II) are each and independently as described above in the first set of values of the variables of Structural Formulae (I) and (II).

A seventeenth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

At least one of A¹, A², A³, A⁸ and A⁹ is -D.

Values of the other variables of Structural Formulae (I) and (II) are each and independently as described above in the first set of values of the variables of Structural Formulae (I) and (II).

An eighteenth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

A¹ is -D; and A², A³, A⁸, and A⁹ are —H.

Values of the other variables of Structural Formulae (I) and (II) are each and independently as described above in the first set of values of the variables of Structural Formulae (I) and (II).

A nineteenth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

A¹, A², A³, A⁸, and A⁹ are -D.

Values of the other variables of Structural Formulae (I) and (II) are each and independently as described above in the first set of values of the variables of Structural Formulae (I) and (II).

A twentieth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

At least one of A¹¹-A²⁰ is -D, and each of A¹-A¹⁰ is —H.

Values of the other variables of Structural Formulae (I) and (II) are each and independently as described above in the first set of values of the variables of Structural Formulae (I) and (II).

A twenty first set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through fifteenth sets of values of the variables of Structural Formulae (I) and (II).

At least one of A¹¹-A²⁰ is -D and at least one of A¹-A¹⁰ is —H.

Values of the other variables of Structural Formulae (I) and (II) are each and independently as described above in the first set of values of the variables of Structural Formulae (I) and (II).

A twenty second set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

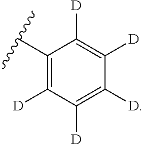

Values of the other variables of Structural Formulae (I) and (II) are each and independently as described above in the first set of values of the variables of Structural Formulae (I) and (II).

A twenty third set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

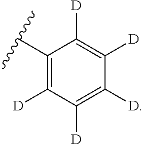

A¹-A²⁰ is —H.

Values of the other variables of Structural Formulae (I) and (II) are each and independently as described above in the first set of values of the variables of Structural Formulae (I) and (II).

A twenty third set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

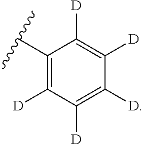

At least one of A¹-A¹⁰ is -D, and each of A¹¹-A²⁰ is —H.

Values of the other variables of Structural Formulae (I) and (II) are each and independently as described above in the first set of values of the variables of Structural Formulae (I) and (II).

A twenty fourth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

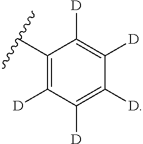

At least one of A¹, A², A³, A⁸ and A⁹ is -D.

Values of the other variables of Structural Formulae (I) and (II) are each and independently as described above in the first set of values of the variables of Structural Formulae (I) and (II).

A twenty fifth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

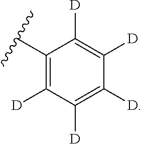

A¹ is -D; and A², A³, A⁸, and A⁹ are —H.

Values of the other variables of Structural Formulae (I) and (II) are each and independently as described above in the first set of values of the variables of Structural Formulae (I) and (II).

A twenty sixth set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

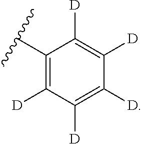

A¹, A², A³, A⁸, and A⁹ are -D.

Values of the other variables of Structural Formulae (I) and (II) are each and independently as described above in the first set of values of the variables of Structural Formulae (I) and (II).

A twenty seventh set of values of the variables of Structural Formulae (I) and (II) is as set forth below:

Each of X, Q, R¹, R³, R⁴, R⁵, R⁶, R⁷, R⁸, R⁹, R^{a}, J^{1A}, J^{9A}, J^{1B}, J^{9B}, J^{1C}, and J^{9C} is independently as described above in any one of the first through thirteenth sets of values of the variables of Structural Formulae (I) and (II).

Y is

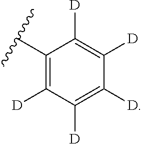

At least one of A¹¹-A²⁰ is -D, and each of A¹-A¹⁰ is —H.

Values of the other variables of Structural Formulae (I) and (II) are each and independently as described above in the first set of values of the variables of Structural Formulae (I) and (II).

In another embodiment, the compounds of the invention are represented by any one of Structural Formulae (III)-(XI):

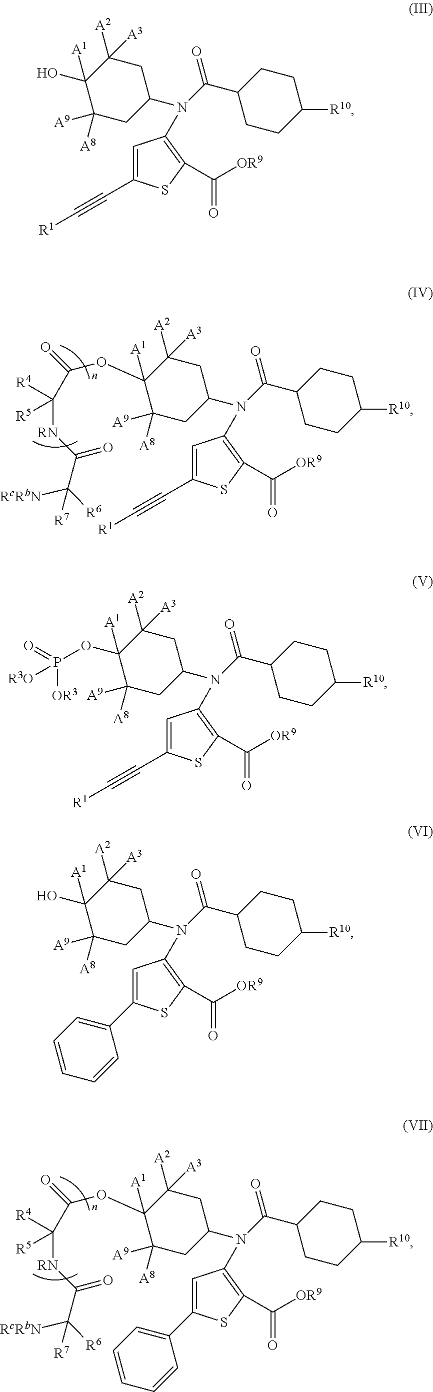

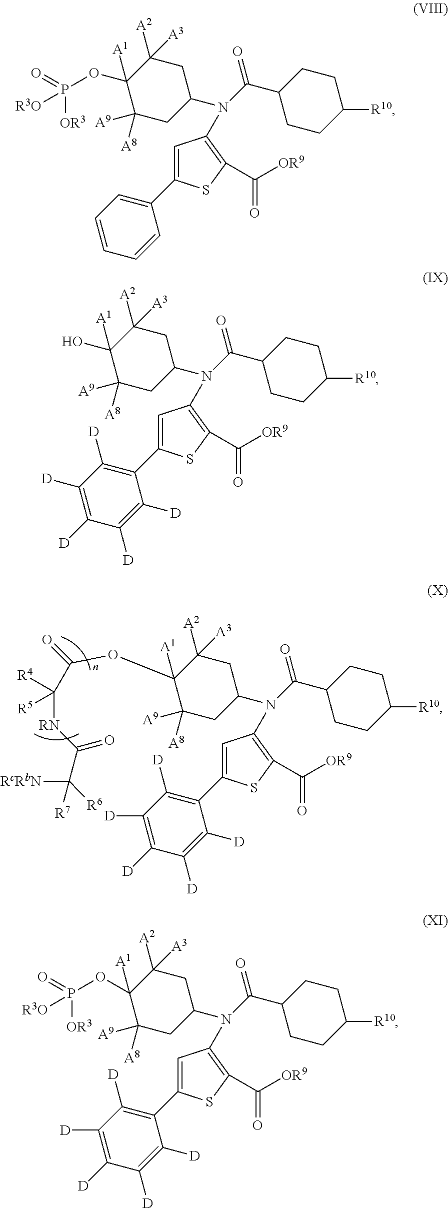

or a pharmaceutically acceptable salt thereof, wherein the first through twenty seventh sets of values of the variables of each Structural Formulae (III)-(VIII), except R¹, A¹, A², A³, A⁸, and A⁹, are each and independently as described above in any one of the first through twenty seventh sets of values of the variables of Structural Formulae (I) and (II)

R¹ is an optionally substituted C₁₋₆ alkyl or optionally substituted C₃-C₈ cycloalkyl.

At least one of A¹, A², A³, A⁸, and A⁹ of each Structural Formulae (III)-(VIII) is -D.

A twenty eighth set of values of the variables of Structural Formulae (III)-(VIII) is as set forth below:

R¹ is an optionally substituted C₁₋₆ alkyl or C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. Specifically, R¹ is C₁₋₆ alkyl or C₃₋₈ cycloalkyl, each of which optionally and independently substituted with one or more substituents selected from the group consisting of halogen, —CN, —OH, —O(C₁₋₆ alkyl), and —O(C₁₋₆ haloalkyl). Specifically, R¹ is C₁₋₆ alkyl or C₃₋₈ cycloalkyl. Specifically, R¹ is t-butyl or isopropyl.

At least one of A¹, A², A³, A⁸, and A⁹ of each Structural Formulae (III)-(VIII) is -D.

Values of the other variables of Structural Formulae (III)-(VIII) are each and independently as described above in any one of the first through twenty seventh sets of values of the variables of Structural Formulae (I) and (II).

A twenty ninth set of values of the variables of Structural Formulae (III)-(VIII) is as set forth below:

R¹, A¹, A², A³, A⁸, and A⁹ are each and independently as described above in the twenty eighth set of values of the variables of Structural Formulae (III)-(VIII).

Each R³ is —H or an optionally substituted C₁₋₆ aliphatic group.

Each of R⁴, R⁵, R⁶ and R² independently —H, or C₁₋₄ alkyl optionally substituted with one or more substituents selected from the group consisting of —OH, —NH₂, —NH—C(═NH)—NH₂, —CO₂H, —C(O)NH₂, phenyl, and hydroxyphenyl.

Each of R^{b} and R^{c} independently is —H or an optionally substituted C₁-C₆ aliphatic group, or optionally, together with the nitrogen atom to which they are attached, form an optionally substituted 4-8 membered heterocyclic ring.

Values of the other variables of Structural Formulae (III)-(VIII) are each and independently as described above in any one of the first through twenty seventh sets of values of the variables of Structural Formulae (I) and (II).

A thirtieth set of values of the variables of the other variables of Structural Formulae (III)-(VIII) is as set forth below:

R¹, A¹, A², A³, A⁸, and A⁹ are each and independently as described above in the twenty eighth set of values of the variables of Structural Formulae (III)-(VIII).

Each R³ independently is —H or C₁₋₆ alkyl.

Each of R⁴, R⁵, R⁶, and R² independently is —H, C₁₋₄ alkyl, —CH₂CO₂H, —CH₂—CH₂—CO₂H, —CH₂—(CH₂)₃—NH₂, —CH₂—(CH₂)₂—NH—C(═NH)—NH₂, —CH₂(phenyl), —CH₂(p-hydroxyphenyl), —CH₂OH, —CH(OH)CH₃, —CH₂C(O)NH₂, or —CH₂CH₂C(O)NH₂.

Each of R^{b} and R^{c} independently is —H or C₁₋₄ alkyl.

Values of the other variables of Structural Formulae (III)-(VIII) are each and independently as described above in any one of the first through twenty seventh sets of values of the variables of Structural Formulae (I) and (II).

A thirty first set of values of the variables of the other variables of Structural Formulae (III)-(VIII) is as set forth below:

R¹, A¹, A², A³, A⁸, and A⁹ are each and independently as described above in the twenty eighth set of values of the variables of Structural Formulae (III)-(VIII).

Each R³, R^{b} and R^{c} is independently is as described above in the thirtieth set of values of the other variables of Structural Formulae (III)-(VIII).

R⁴ and R⁶ are each independently —H or C₁₋₆ alkyl; R⁵ and R² are each independently —H or optionally substituted C₁₋₆ alkyl. Alternatively, each of R⁴, R⁵, R⁶, and R² independently is —H, or C₁₋₄ alkyl.

Values of the other variables of Structural Formulae (III)-(VIII) are each and independently as described above in any one of the first through twenty seventh sets of values of the variables of Structural Formulae (I) and (II).

A thirty second set of values of the variables of the other variables of Structural Formulae (III)-(VIII) is as set forth below:

R¹, R³, R⁴, R⁵, R⁶, R², R^{b}, R^{c}, A¹, A², A³, A⁸, and A⁹ are each and independently as described above in any one of the twenty eighth through thirty first sets of values of the variables of Structural Formulae (III)-(VIII).

R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —OC(O)O(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), phenyl, and 5-6 membered heterocycle optionally substituted with one or more substituents selected from the group consisting of oxo and C₁₋₆alkyl. Specifically, R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl)-CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. More specifically, R⁹ is —H.

Values of the other variables of Structural Formulae (III)-(VIII) are each and independently as described above in any one of the first through twenty seventh sets of values of the variables of Structural Formulae (I) and (II).

In yet another embodiment, the compounds of the invention are represented by any one of Structural Formulae (XII), (XIII), and (XIV):

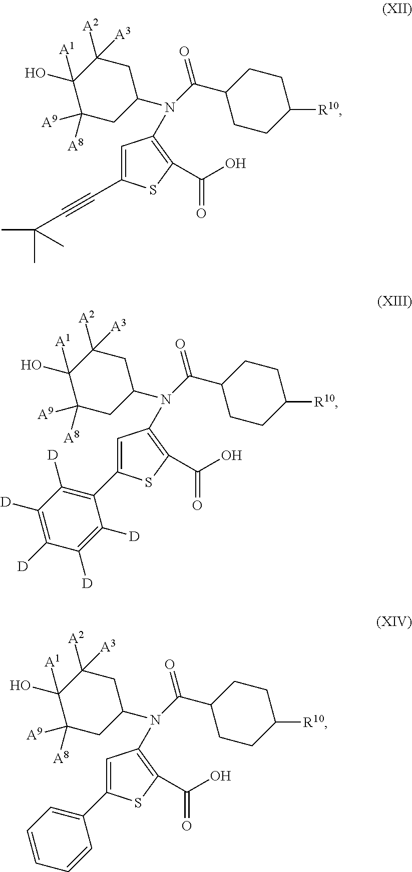

or a pharmaceutically acceptable salt thereof, wherein:

At least one of A¹, A², A³, A⁸, and A⁹ of each Structural Formula (XII) and (XIV) is -D. Specifically, A¹ is -D; and A², A³, A⁸, and A⁹ are —H. Specifically, A¹, A², A³, A⁸, and A⁹ are -D.

Values of the other variables Structural Formulae (XII), (XIII), and (XIV) are each and independently as described above in the first through thirty second sets of the variables of Structural Formulae (III)-(VIII).

In another set of values of the variables Structural Formulae (XII), (XIII), and (XIV):

At least one of A¹, A², A³, A⁸, and A⁹ of each Structural Formula (XII) and (XIV) is -D. Specifically, A¹ is -D; and A², A³, A⁸, and A⁹ are —H. Specifically, A¹, A², A³, A⁸, and A⁹ are -D.

R¹⁰ is —CH₃.

Values of the other variables Structural Formulae (XII), (XIII), and (XIV) are each and independently as described above in the first through thirty second sets of the variables of Structural Formulae (III)-(VIII).

In yet another embodiment, the compounds of the invention are represented by any one of Structural Formulae (XV), (XVI), and (XVII):

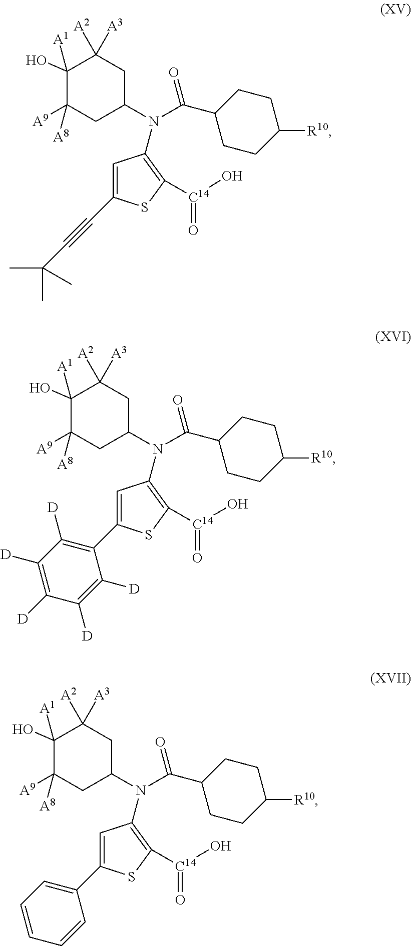

or a pharmaceutically acceptable salt thereof, wherein:

Each of A¹, A², A³, A⁸, and A⁹ independently is —H or -D. Specifically, A¹ is -D; and A², A³, A⁸, and A⁹ are —H. Specifically, A¹, A², A³, A⁸, and A⁹ are -D. Specifically, A¹, A², A³, A⁸, and A⁹ are —H.

Values of the other variables Structural Formulae (XV), (XVI), and (XVII) are each and independently as described above in the first through thirty second sets of the variables of Structural Formulae (III)-(VIII).

In another set of values of the variables Structural Formulae ((XV), (XVI), and (XVII):

Each of A¹, A², A³, A⁸, and A⁹ independently is —H or -D. Specifically, A¹ is -D; and A², A³, A⁸, and A⁹ are —H. Specifically, A¹, A², A³, A⁸, and A⁹ are -D. Specifically, A¹, A², A³, A⁸, and A⁹ are —H.

R¹⁰ is —CH₃.

Values of the other variables Structural Formulae (XV), (XVI), and (XVII) are each and independently as described above in the first through thirty second sets of the variables of Structural Formulae (III)-(VIII).

In yet another embodiment, the compounds of the invention are pharmaceutically acceptable salts of any one of Structural Formulae (I)-(XVII), wherein the values of the variables are each and independently as described above.

In yet another embodiment, a compound of the invention is selected compound selected from the structural formulae depicted below:

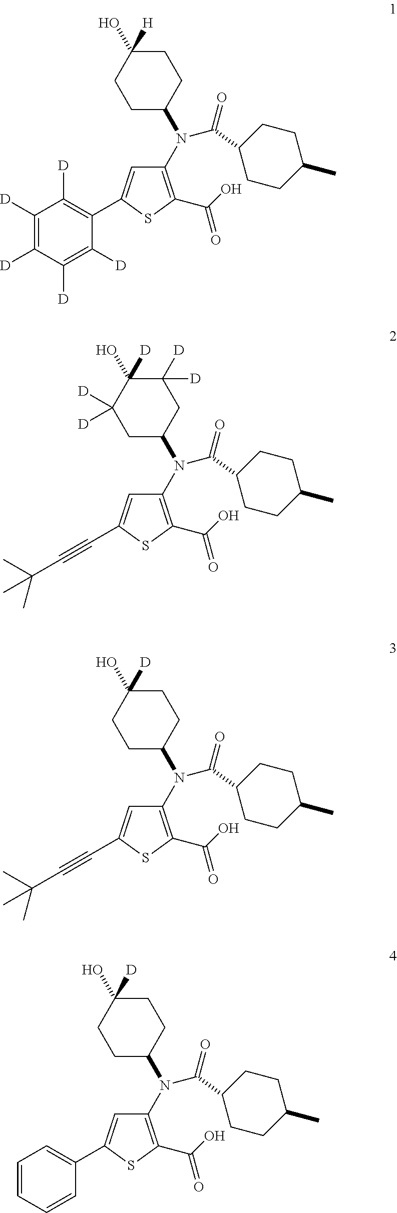

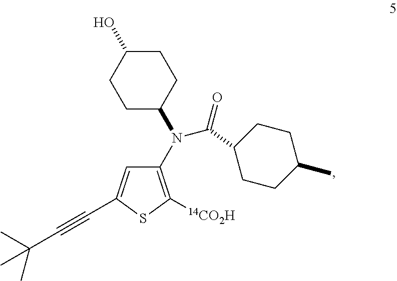

or a pharmaceutically acceptable salt thereof.

As used herein, a reference to compound(s) of the invention, for example compound(s) of Structural Formula (I), or compound(s) of claim 1, will also include pharmaceutically acceptable salts thereof.

The compounds according to the invention described herein can be prepared by any suitable method known in the art. For example, the compounds can be prepared in accordance with procedures described in U.S. Pat. No. 6,881,741, US 2005/0009804, US 2006/0276533, WO 2002/100851, and WO 08/58393, the disclosures of which are hereby incorporated by reference.

In one embodiment, the compounds of the invention (e.g., compounds of Structural Formulae (I)-(XVIII)) can be prepared as depicted in General Schemes 1-11. For example, the compounds of Structural Formulae (I)-(XI) can be prepared as shown in General Schemes 1-11, respectively. Any suitable condition known in the art can be employed for each step described in the schemes. Specific exemplary conditions are described in the schemes, and exemplary detailed procedures are described below in the Exemplification section.

In a specific embodiment, the present invention provides methods of preparing a compound represented by Structural Formula (I). The methods comprise the step of reducing compound (1 h) or compound (1k) (by the reduction of its ketone group) with a suitable reducing agent, for example, NaB(A¹)₄, to form compound (1i), a compound of Structural Formula (I) where X is —H, and R⁹ is -Me. The reduced compound (1i), if desired, can then optionally further be hydrolyzed to from compound (lj), a compound of Structural Formula (I) where X is —H and R⁹ is —H. Optionally, if desired, compound (lj) can further be reacted with HO—[C(O)C(R⁴R⁵)N(R)]ₙ—C(O)C(R⁶R⁷)NR^{b}R^{c} for the compounds of Structural Formula (I) having [—C(O)C(R⁴R⁵)N(R)-]ₙ—C(O)C(R⁶R⁷)NR^{b}R^{c} for X; or with (R^{k})₂N—P(OR³)₂ (where R^{k} is typically alkyl (e.g., ethyl), benzyl, etc.) for the compounds of Structural Formula (I) having —P(O)(OR³)₂ for X; or with HOC(O)R² for the compounds of Structural Formula (I) having —C(O)R² for X. Alternatively, the compounds of Structural Formula (I) can be prepared via compound (1k) by the reduction of its ketone group by a suitable reducing agent, for example, NaB(A¹)₄. Optionally, A², A³, A⁸ and A⁹ can be introduced, as desired, by the reaction with MeOAⁿ in Aⁿ₂O where Aⁿ is A², A³, A⁸ or A⁹. If desired, compound (1j) (a compound of Structural Formula (I) where X is —H, and R⁹ is -Me) can be further reacted with a suitable reagent(s) known in the art to form compounds having other than —H for R⁹.

The compounds described in General Scheme 1, including compounds (1a), (1c), (1e), (1f), (1g), (1 h), (1i), (1j), and (1k), can generally be prepared by any suitable method known in the art. In a specific embodiment, the methods further comprise the step of preparing compound (1 h) or (1k), as described in General Scheme 1. Particularly, reaction of compound (1f) with YH for Y is —C≡CR¹, or with R¹YB(OR^{k})₂ (where R^{k} is typically —H, C₁₋₆ alkyl (e.g., Me or Et), or benzyl) for Y is phenylene (—C₆H₅) or d5-phenylene (—C₆D₅) can produce compound (1g). Subsequent treatment of compound (1g) with an acid (e.g., HCl) in an aqueous condition can produce compound (1 h).

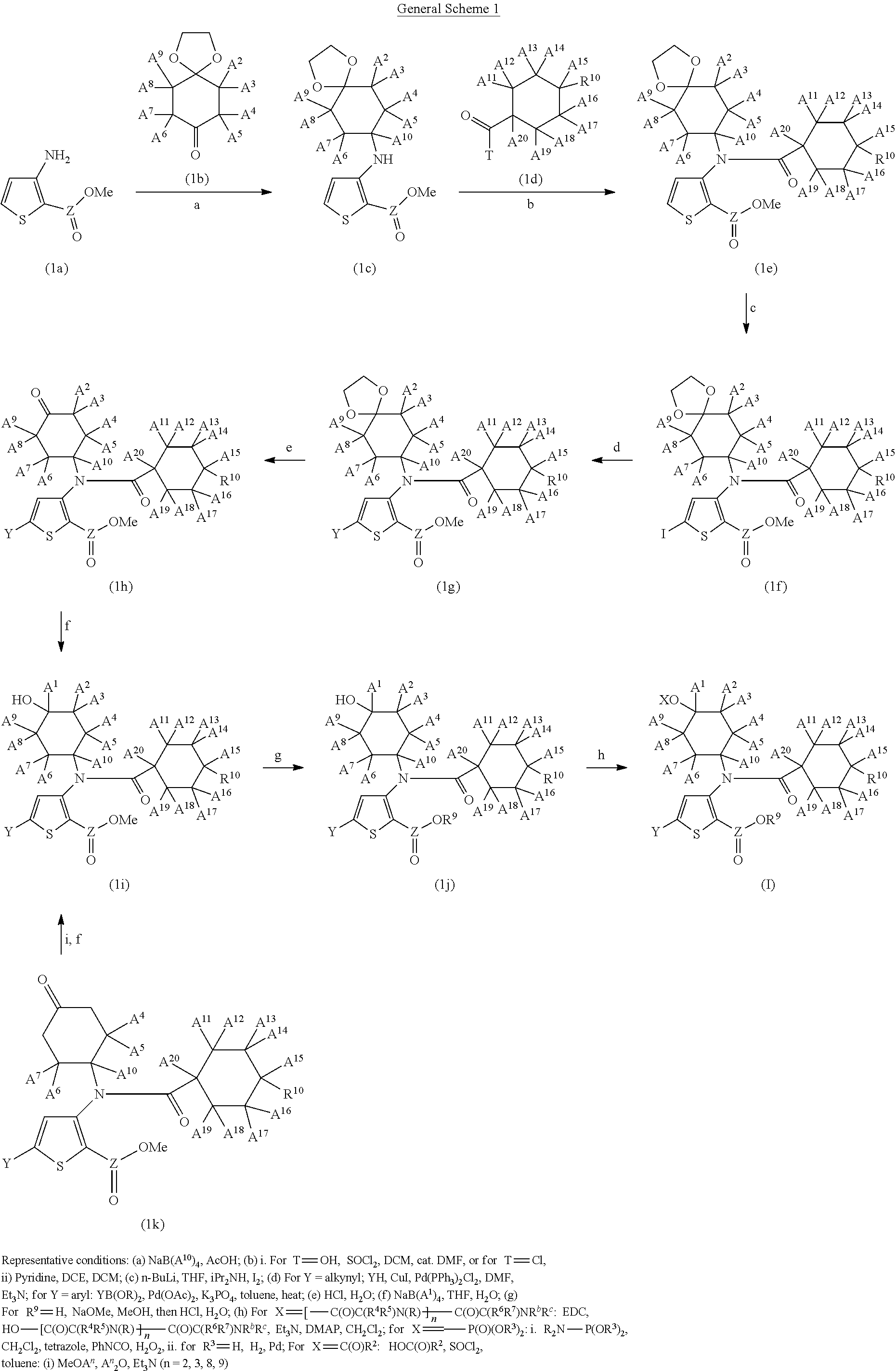

In another specific embodiment, the methods are as described in each of General Schemes 2 and 3. General Schemes 2 and 3 show general synthetic schemes for the compounds of Structural Formulae (II) and (III), respectively. The synthetic details are each and independently as described above for General Scheme 1. For example, compounds (2a)-(2j), and compounds (3b)-(3i) described in those schemes are each independently as described in General Scheme 1 for compounds (1a)-(1i).

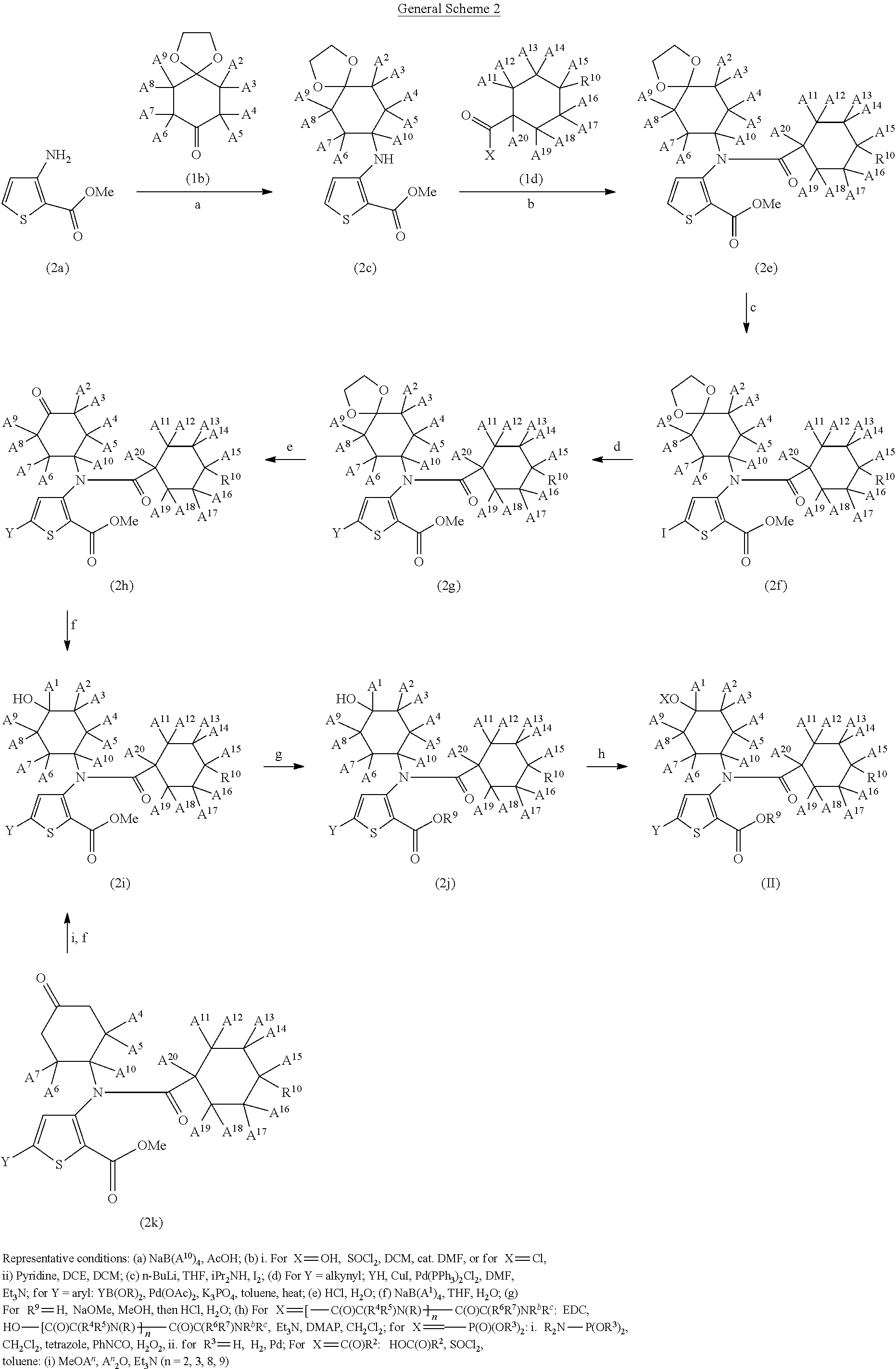

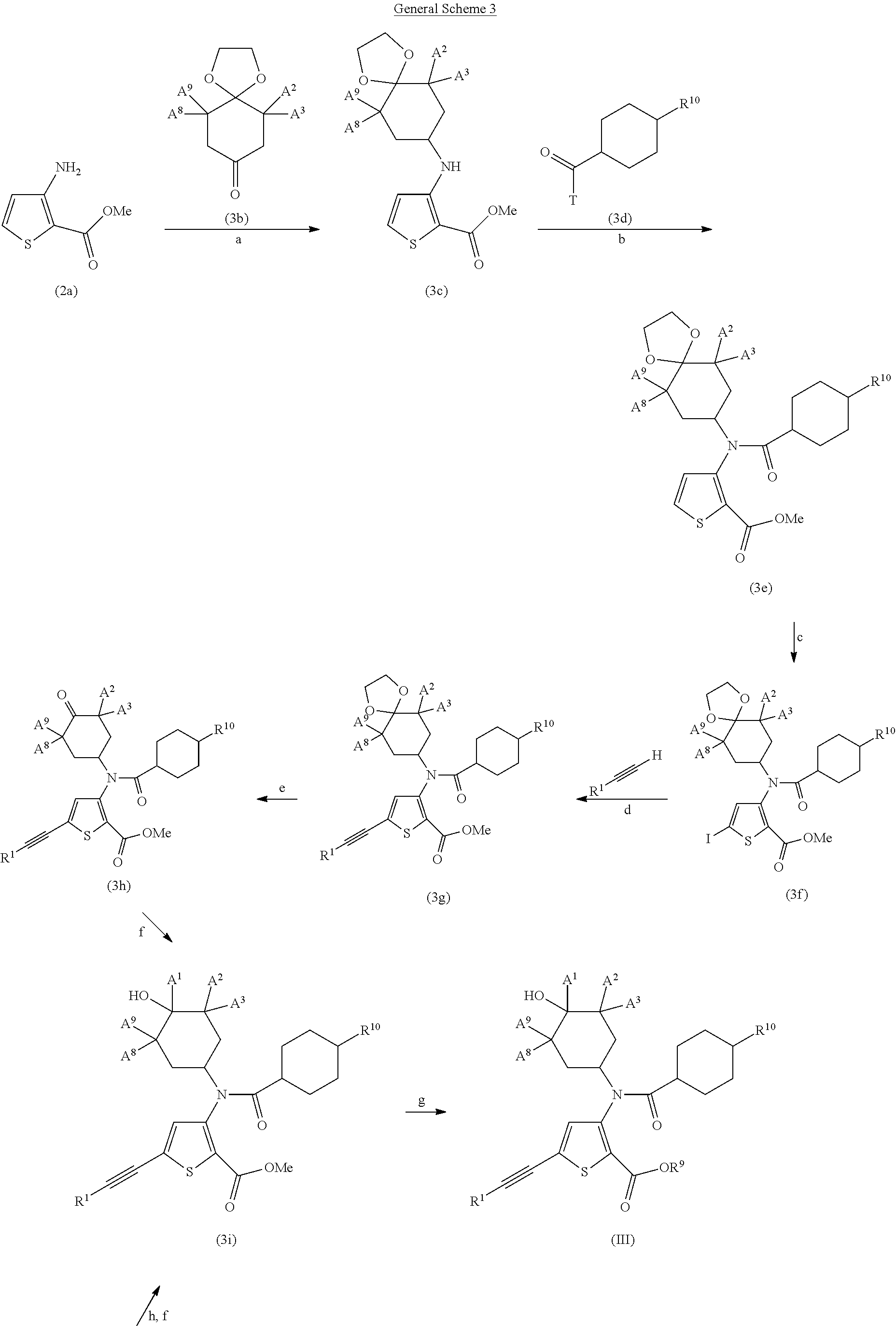

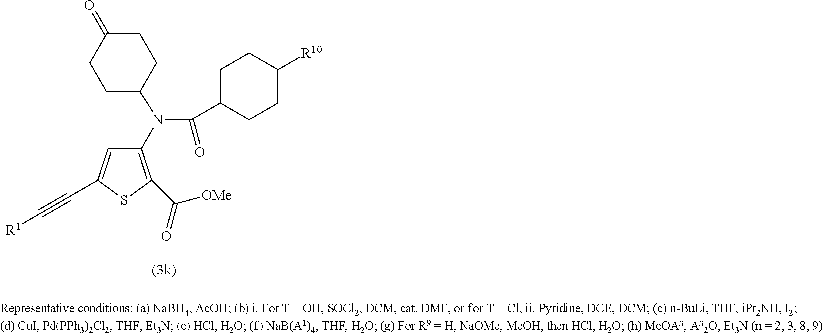

In yet another specific embodiment, the methods are as described in each of General Schemes 4 and 5. General Schemes 4 and 5 show general synthetic schemes for the compounds of Structural Formula (IV) and (V), respectively. The synthetic details are each and independently as described above for General Scheme 1. For example, the compounds of Structural Formula (IV) can be prepared from a compound of Structural Formula (III) by the reaction with HO—[C(O)C(R⁴R⁵)N(R)]ₙC(O)C(R⁶R⁷)NR^{b}R^{c} under a suitable condition; and the compounds of Structural Formula (V) can be prepared from a compound of Structural Formula (III) by the reaction with (R^{k})₂N—P(OR³)₂ (wherein R^{k} is typically —H, C₁₋₆ alkyl (e.g., ethyl), benzyl, etc.) under a suitable condition.

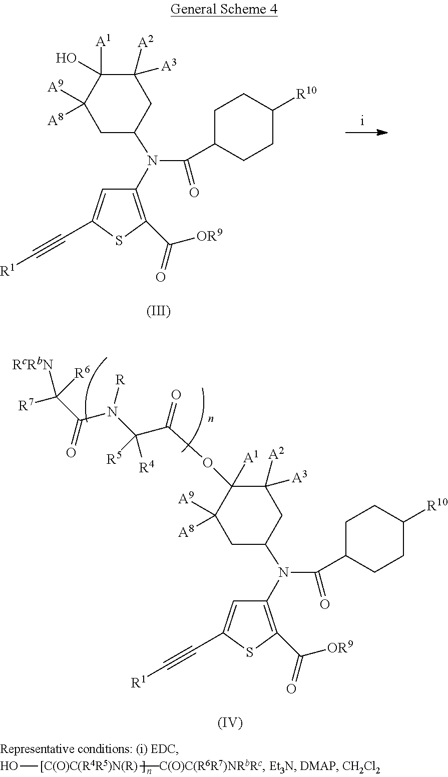

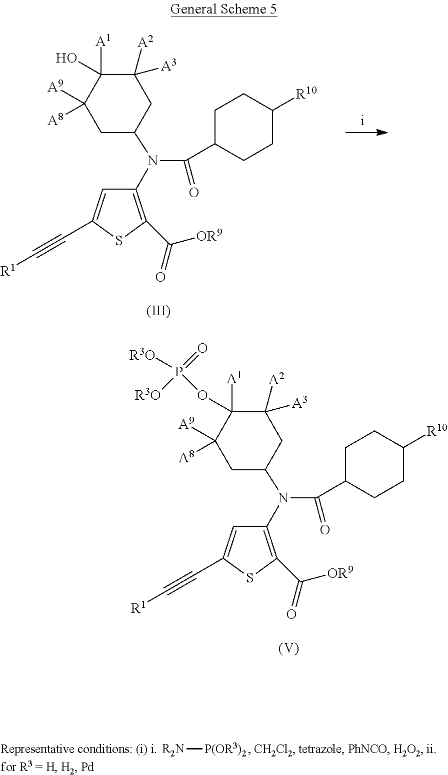

In yet another specific embodiment, the methods are as described in General Scheme 6. General Scheme 6 shows a general synthetic scheme for the compounds of Structural Formula (VI). The synthetic details are each and independently as described above for General Scheme 1. For example, compounds (2a), (3b), (3d), (6c)-(6k) are each independently as described in General Scheme 1 for compounds (1a)-(1k).

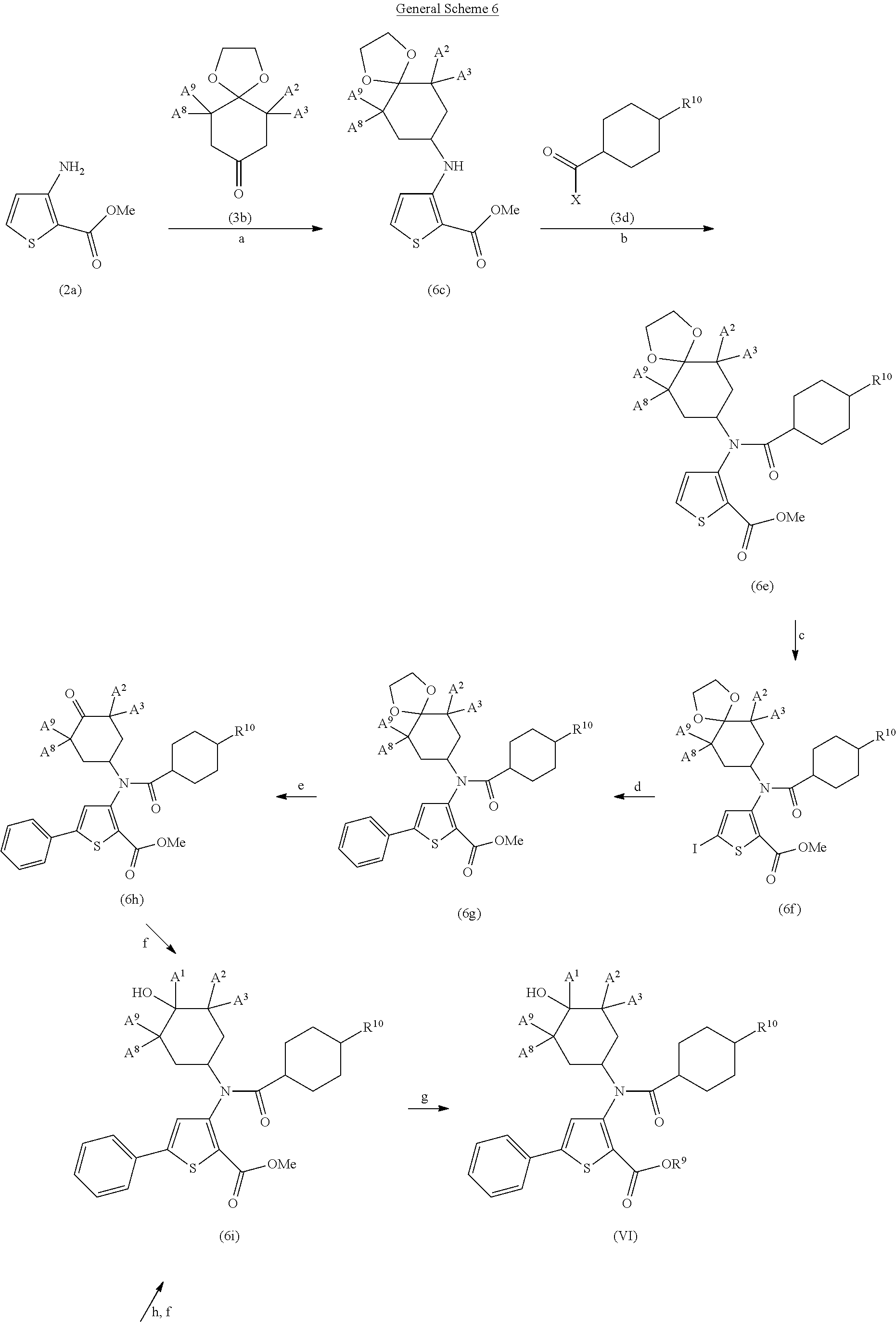

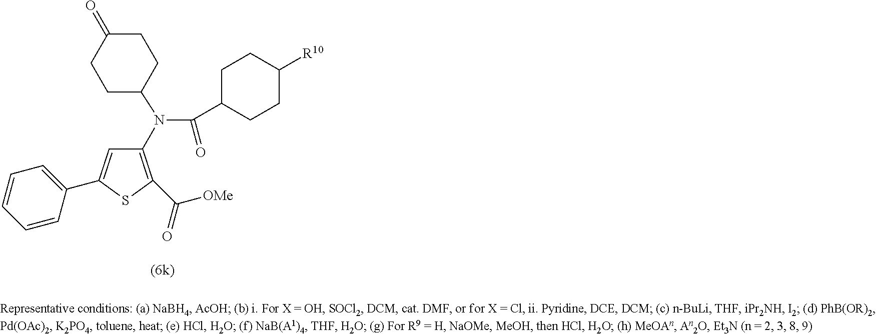

In yet another specific embodiment, the methods are as described in each of General Schemes 7 and 8. General Schemes 7 and 8 show general synthetic schemes for the compounds of Structural Formula (VII) and (VIII), respectively. The synthetic details are each and independently as described above for General Scheme 1. For example, the compounds of Structural Formula (VII) can be prepared from a compound of Structural Formula (VI) by the reaction with HO—[C(O)C(R⁴R⁵)N(R)]ₙC(O)C(R⁶R⁷)NR^{b}R^{c} under a suitable condition; and the compounds of Structural Formula (VIII) can be prepared from a compound of Structural Formula (VI) by the reaction with (R^{k})₂N—P(OR³)₂ (wherein R^{k} is typically —H, C₁₋₆ alkyl (e.g., ethyl), benzyl, etc.) under a suitable condition.

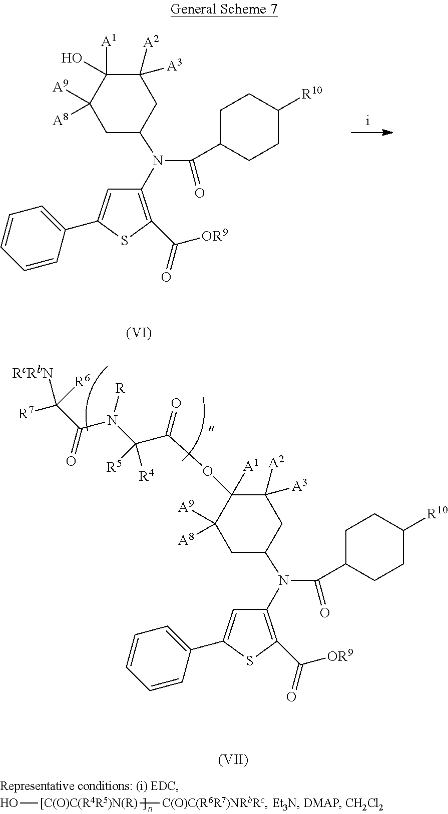

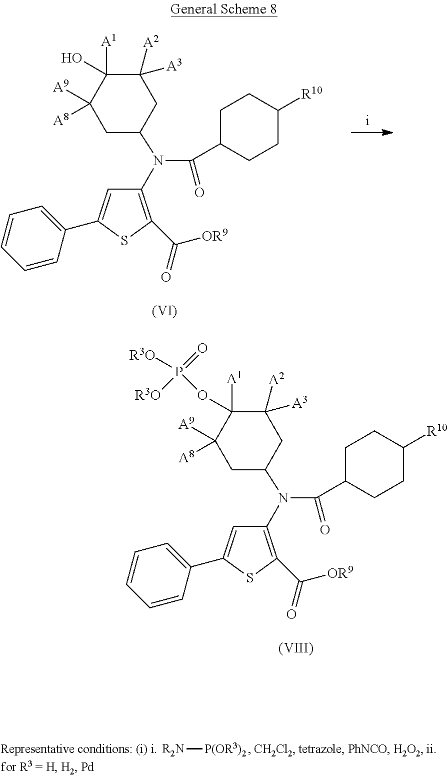

In yet another specific embodiment, the methods are as described in General Scheme 9. General Scheme 9 shows a general synthetic scheme for the compounds of Structural Formula (IX). The synthetic details are each and independently as described above for General Scheme 1. For example, compounds (3e), (9f), (9g), (9h), and (9k) are each independently as described in General Scheme 1 for compounds (3e), and (1f)-(1k).

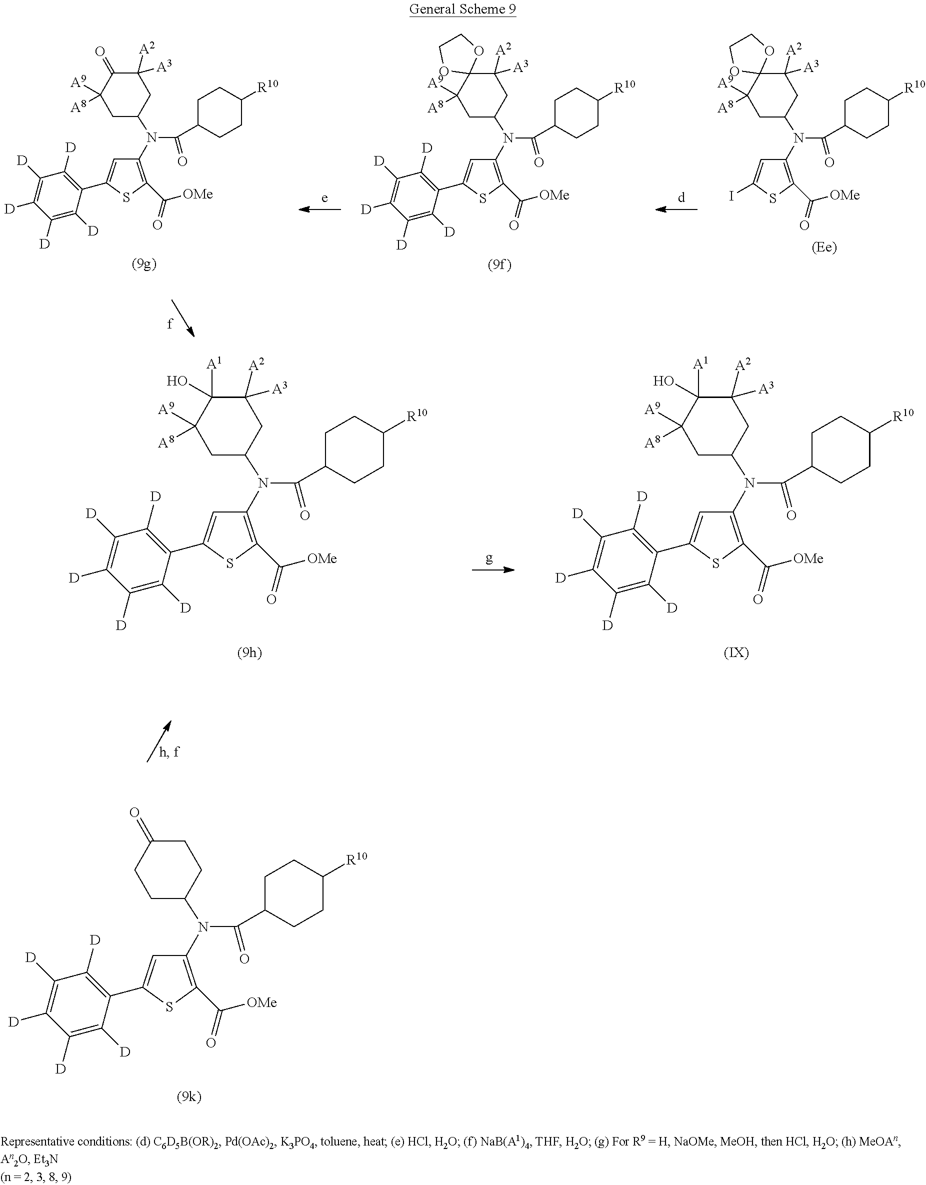

In yet another specific embodiment, the methods are as described in each of General Schemes 10 and 11. General Schemes 10 and 11 show general synthetic schemes for the compounds of Structural Formula (X) and (XI), respectively. The synthetic details are each and independently as described above for General Scheme 1. For example, the compounds of Structural Formula (X) can be prepared from a compound of Structural Formula (IX) by the reaction with HO—[C(O)C(R⁴R⁵)N(R)]ₙC(O)C(R⁶R⁷)NR^{b}R^{c} under a suitable condition; and the compounds of Structural Formula (XI) can be prepared from a compound of Structural Formula (IX) by the reaction with (R^{k})₂N—P(OR³)₂ (wherein R^{k} is typically —H, C₁₋₆ alkyl (e.g., ethyl), benzyl, etc.) under a suitable condition.

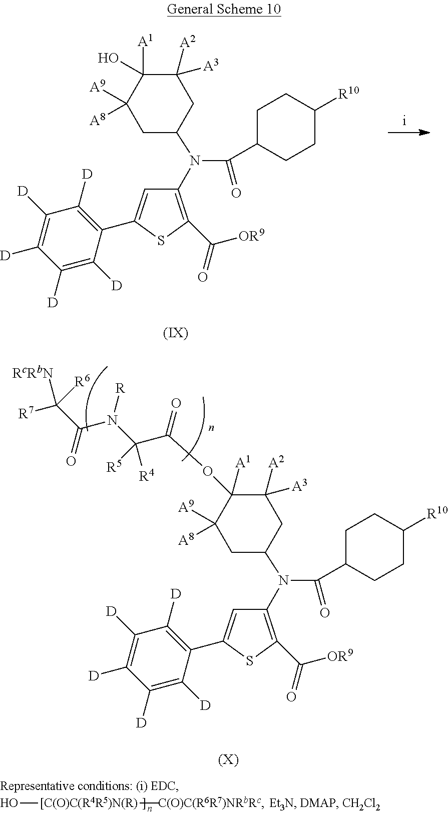

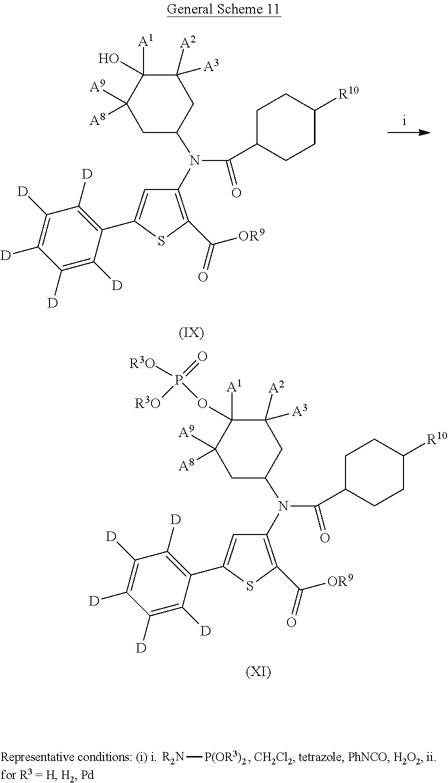

In some embodiments, the compounds of the invention are represented by any one of Structural Formulae (I)-(V) or pharmaceutically acceptable salts thereof, wherein R¹ is —C≡C(CD₃)₃. Such compounds can be prepared as described above, for example, as described in General Schemes 1-5, wherein compounds (1f), (2f), and (3f) each and independently react with HC≡C(CD₃)₃. In some specific embodiments, the compounds of the invention are represented by Structural Formula (XVIII) or pharmaceutically acceptable salts thereof:

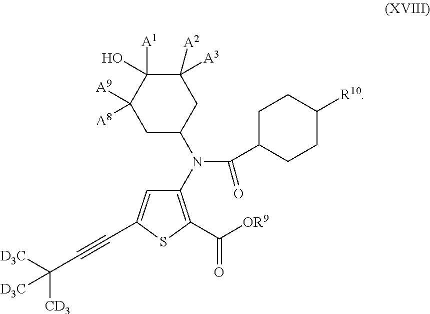

It will be appreciated by those skilled in the art that in the processes of the present invention certain functional groups such as hydroxyl or amino groups in the starting reagents or intermediate compounds may need to be protected by protecting groups. Thus, the preparation of the compounds described above may involve, at various stages, the addition and removal of one or more protecting groups. The protection and deprotection of functional groups is described in “Protective Groups in Organic Chemistry.” edited by J. W. F. McOmie, Plenum Press (1973) and “Protective Groups in Organic Synthesis,” 3rd edition, T. W. Greene and P. G. M. Wuts, Wiley Interscience, and “Protecting Groups,” 3rd edition, P. J. Kocienski, Thieme (2005)

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as illustrated generally below, or as exemplified by particular classes, subclasses, and species of the compounds described above. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. For example, if X is optionally substituted C₁-C₃alkyl or phenyl; X may be either optionally substituted C₁-C₃ alkyl or optionally substituted phenyl. Likewise, if the term “optionally substituted” follows a list, said term also refers to all of the substitutable groups in the prior list unless otherwise indicated. For example: if X is C₁-C₃alkyl or phenyl wherein X is optionally and independently substituted by J^{X}, then both C₁-C₃alkyl and phenyl may be optionally substituted by J^{X}. As is apparent to one having ordinary skill in the art, groups such as H, halogen, NO₂, CN, NH₂, OH, or OCF₃ would not be substitutable groups.

The phrase “up to”, as used herein, refers to zero or any integer number that is equal or less than the number following the phrase. For example, “up to 3” means any one of 0, 1, 2, and 3. As described herein, a specified number range of atoms includes any integer therein. For example, a group having from 1-4 atoms could have 1, 2, 3, or 4 atoms.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “aliphatic” or “aliphatic group”, as used herein, means a straight-chain (i.e., unbranched), or branched, hydrocarbon chain that is completely saturated or that contains one or more units of unsaturation but is non-aromatic. Unless otherwise specified, aliphatic groups contain 1-10 aliphatic carbon atoms. In some embodiments, aliphatic groups contain 1-6 aliphatic carbon atoms. In other embodiments, aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl and acetylene.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds. Each of the “alkyl”, “alkenyl” or “alkynyl” as used herein can be optionally substituted as set forth below. In some embodiments, the “alkyl” is C₁-C₆ alkyl or C₁-C₄ alkyl. In some embodiments, the “alkenyl” is C₂-C₆ alkenyl or C₂-C₄ alkenyl. In some embodiments, the “alkynyl” is C₂-C₆ alkynyl or C₂-C₄ alkynyl.

The term “cycloaliphatic” (or “carbocycle” or “carbocyclyl” or “carbocyclic”) refers to a non-aromatic carbon only containing ring system which can be saturated or contains one or more units of unsaturation, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged carbocyclic ring systems. The term also includes polycyclic ring systems in which the carbocyclic ring can be “fused” to one or more non-aromatic carbocyclic or heterocyclic rings or one or more aromatic rings or combination thereof, wherein the radical or point of attachment is on the carbocyclic ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl,” or “heterocyclic” or “non-aromatic heterocycle”) as used herein refers to a non-aromatic ring system which can be saturated or contain one or more units of unsaturation, having three to fourteen ring atoms in which one or more ring carbons is replaced by a heteroatom such as, N, S, or O. In some embodiments, non-aromatic heterocyclic rings comprise up to three heteroatoms selected from N, S and O within the ring. In other embodiments, non-aromatic heterocyclic rings comprise up to two heteroatoms selected from N, S and O within the ring system. In yet other embodiments, non-aromatic heterocyclic rings comprise up to three heteroatoms selected from N and O within the ring system. In yet other embodiments, non-aromatic heterocyclic rings comprise up to two heteroatoms selected from N and O within the ring system. The term includes monocyclic, bicyclic or polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl.

The term “aryl” (or “aryl ring” or “aryl group”) used alone or as part of a larger moiety as in “aralkyl”, “aralkoxy”, “aryloxyalkyl”, or “heteroaryl” refers to carbocyclic aromatic ring systems. The term “aryl” may be used interchangeably with the terms “aryl ring” or “aryl group”. “Carbocyclic aromatic ring” groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring” or “carbocyclic aromatic”, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The terms “heteroaryl”, “heteroaromatic”, “heteroaryl ring”, “heteroaryl group”, “aromatic heterocycle” or “heteroaromatic group”, used alone or as part of a larger moiety as in “heteroaralkyl” or “heteroarylalkoxy”, refer to heteroaromatic ring groups having five to fourteen members, in which one or more ring carbons is replaced by a heteroatom such as, N, S, or O. In some embodiments, heteroaryl rings comprise up to three heteroatoms selected from N, S and O within the ring. In other embodiments, heteroaryl rings comprise up to two heteroatoms selected from N, S and O within the ring system. In yet other embodiments, heteroaryl rings comprise up to three heteroatoms selected from N and O within the ring system. In yet other embodiments, heteroaryl rings comprise up to two heteroatoms selected from N and O within the ring system. Heteroaryl rings include monocyclic heteroaromatic rings and polycyclic aromatic rings in which a monocyclic aromatic ring is fused to one or more other aromatic rings. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo”, “cyclic”, “cyclic group” or “cyclic moiety”, include mono-, bi-, and tri-cyclic ring systems including cycloaliphatic, heterocycloaliphatic, aryl, or heteroaryl, each of which has been previously defined.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “bridged bicyclic ring system” refers to a bicyclic heterocycloalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “bridge” refers to a bond or an atom or an unbranched chain of atoms connecting two different parts of a molecule. The two atoms that are connected through the bridge (usually but not always, two tertiary carbon atoms) are denotated as “bridgeheads”.

As used herein, the term “spiro” refers to ring systems having one atom (usually a quaternary carbon) as the only common atom between two rings.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein an optionally substituted aralkyl can be substituted on both the alkyl and the aryl portion. Unless otherwise indicated as used herein optionally substituted aralkyl is optionally substituted on the aryl portion.

In some embodiments, an aliphatic group and a heterocyclic ring may independently contain one or more substituents. Suitable substituents on the saturated carbon of an aliphatic group or of a non-aromatic heterocyclic ring are selected from those described above. Other suitable substitutents include those listed as suitable for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, wherein each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

In some embodiments, optional substituents on the nitrogen of a heterocyclic ring include those described above. Examples of such suitable substituents include —OH, —NH₂, —NH(C₁-C₄ alkyl), —N(C₁-C₄ alkyl)₂, —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

In some embodiments, an aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from those described above. Specific examples include halogen, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include: halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(S)R^{o}; —NR^{o}C(O)N(R^{o})₂; —NR^{o}C(S)N(RO₂; —NR^{o}CO₂R^{o}; —NR^{o}NR^{o}C(O)R^{o}; —NR^{o}NR^{o}C(O)N(RO₂; —NR^{o}NR^{o}CO₂R^{o}; —C(O)C(O)R^{o}; —C(O)CH₂C(O)R^{o}; —CO₂R^{o}; —C(O)R^{o}; —C(S)R^{o}; —C(O)N(R^{o})₂; —C(S)N(R^{o})₂; —OC(O)N(R^{o})₂; —OC(O)R^{o}; —C(O)N(OR^{o}R^{o}; —C(NOR^{o}R^{o}; —S(O)₂R^{o}; —S(O)₃R^{o}; —SO₂N(R^{o})₂; —S(O)R^{o}; —NR^{o}SO₂N(R^{o})₂; —NR^{o}SO₂R^{o}; —N(OR^{o}R^{o}; —C(═NH)—N(R^{o})₂; or —(CH₂)₀₋₂NHC(O)R^{o}; wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, two independent occurrences of R^{o}, on the same substituent or different substituents, taken together with the atom(s) to which each R^{o} group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄ aliphatic), O(haloC₁₋₄ aliphatic), or haloC₁₋₄aliphatic, CHO, N(CO)(C₁₋₄ aliphatic), C(O)N(C₁₋₄ aliphatic), wherein each of the foregoing C₁₋₄aliphatic groups of R^{o} is unsubstituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

The term “unsaturated”, as used herein, means that a moiety has one or more units of unsaturation.

As detailed above, in some embodiments, two independent occurrences of R^{o} (or R⁺, or any other variable similarly defined herein), may be taken together with the atom(s) to which each variable is bound to form a 5-8-membered heterocyclyl, aryl, or heteroaryl ring or a 3-8-membered cycloalkyl ring. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of

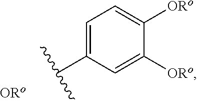

these two occurrences of R^{o} are taken together with the oxygen atoms to which they are bound to form a fused 6-membered oxygen containing ring:

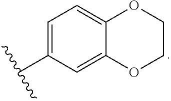 [SMILES: *c1ccc2c(c1)OCCO2]

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

As used herein, an “amino” group refers to —NH₂.

The term “hydroxyl” or “hydroxy” or “alcohol moiety” refers to —OH.

As used herein, an “oxo” refers to ═O.

As used herein, the term “alkoxy”, or “alkylthio”, as used herein, refers to an alkyl group, as previously defined, attached to the molecule through an oxygen (“alkoxy” e.g., —O-alkyl) or sulfur (“alkylthio” e.g., —S-alkyl) atom.

As used herein, the terms “halogen”, “halo”, and “hal” mean F, Cl, Br, or I.

As used herein, the term “cyano” or “nitrile” refer to —CN or —CN.

The terms “alkoxyalkyl”, “alkoxyalkenyl”, “alkoxyaliphatic”, and “alkoxyalkoxy” mean alkyl, alkenyl, aliphatic or alkoxy, as the case may be, substituted with one or more alkoxy groups.

The terms “haloalkyl”, “haloalkenyl”, “haloaliphatic”, “haloalkoxy”, and “cyclo(haloalkyl)” mean alkyl, alkenyl, aliphatic, alkoxy, or cycloalkyl, as the case may be, substituted with one or more halogen atoms. This term includes perfluorinated alkyl groups, such as —CF₃ and —CF₂CF₃.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. In some embodiments, the cyanoalkyl is (NC)-alkyl-.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups, wherein the amino group is as defined above.

The terms “hydroxyalkyl”, “hydroxyaliphatic”, and “hydroxyalkoxy” mean alkyl, aliphatic or alkoxy, as the case may be, substituted with one or more —OH groups.

The terms “alkoxyalkyl”, “alkoxyaliphatic”, and “alkoxyalkoxy” mean alkyl, aliphatic or alkoxy, as the case may be, substituted with one or more alkoxy groups. For example, an “alkoxyalkyl” refers to an alkyl group such as (alkyl—O)-alkyl-, wherein alkyl has been defined above.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

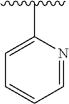 [SMILES: *c1ccccn1]

also represents

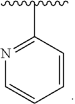 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful. For example, the compounds represented by Structural Formula (XVIII) below are also within the scope of this invention:

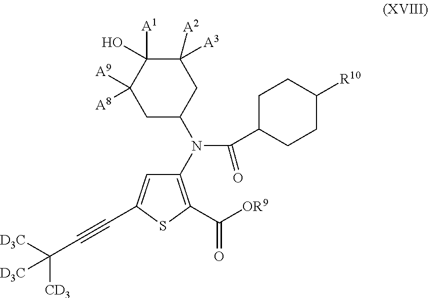

where the variables of Structural Formula (XVIII) are each and independently as described above.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds described herein can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by, for example, 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by, for example, 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds described herein, the methods of the invention can be employed for preparing pharmaceutically acceptable solvates (e.g., hydrates) and clathrates of these compounds.

As used herein, the term “pharmaceutically acceptable solvate,” is a solvate formed from the association of one or more pharmaceutically acceptable solvent molecules to one of the compounds described herein. The term solvate includes hydrates (e.g., hemihydrate, monohydrate, dihydrate, trihydrate, tetrahydrate, and the like).

As used herein, the term “hydrate” means a compound described herein or a salt thereof that further includes a stoichiometric or non-stoichiometric amount of water bound by non-covalent intermolecular forces.

As used herein, the term “clathrate” means a compound described herein or a salt thereof in the form of a crystal lattice that contains spaces (e.g., channels) that have a guest molecule (e.g., a solvent or water) trapped within.

In addition to the compounds described herein, the methods of the invention can be employed for preparing pharmaceutically acceptable derivatives or prodrugs of these compounds.

A “pharmaceutically acceptable derivative or prodrug” includes any pharmaceutically acceptable ester, salt of an ester, or other derivative or salt thereof, of a compound described herein, which, upon administration to a recipient, is capable of providing, either directly or indirectly, a compound described herein or an inhibitorily active metabolite or residue thereof. Particularly favoured derivatives or prodrugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species.

As used herein and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties. Prodrugs can typically be prepared using well-known methods, such as those described by BURGER′S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

Pharmaceutically acceptable prodrugs of the compounds described above include, without limitation, esters, amino acid esters, phosphate esters, metal salts and sulfonate esters.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds of the invention are provided in the form of a single enantiomer at least 95%, at least 97% and at least 99% free of the corresponding enantiomer.

In a further embodiment, the compounds of the invention are in the form of the (+) enantiomer at least 95% free of the corresponding (−) enantiomer.

In a further embodiment, the compounds of the invention are in the form of the (+) enantiomer at least 97% free of the corresponding (−) enantiomer.

In a further embodiment, the compounds of the invention are in the form of the (+) enantiomer at least 99% free of the corresponding (−) enantiomer.

In a further embodiment, the compounds of the invention are in the form of the (−) enantiomer at least 95% free of the corresponding (+) enantiomer.

In a further embodiment, the compounds of the invention are in the form of the (−) enantiomer at least 97% free of the corresponding (+) enantiomer.

In a further embodiment the compounds of the invention are in the form of the (−) enantiomer at least 99% free of the corresponding (+) enantiomer.

In some embodiments, the compounds of the invention are provided as pharmaceutically acceptable salts. As discussed above, such pharmaceutically acceptable salts can be derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄_{⁺} (where R is C₁₋₄ alkyl) salts, choline and tromethamine.

In one embodiment of the invention, the pharmaceutically acceptable salt is a sodium salt.

In one embodiment of the invention, the pharmaceutically acceptable salt is a potassium salt.

In one embodiment of the invention, the pharmaceutically acceptable salt is a lithium salt.

In one embodiment of the invention, the pharmaceutically acceptable salt is a tromethamine salt.

In one embodiment of the invention, the pharmaceutically acceptable salt is an L-arginine salt.

It will be appreciated by those skilled in the art that the compounds of the invention described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds of the invention described herein can exist in different solvate forms, for example hydrates. Solvates of the compounds of the invention may also form when solvent molecules are incorporated into the crystalline lattice structure of the compound molecule during the crystallization process.

The terms “subject,” “host,” or “patient” includes an animal and a human (e.g., male or female, for example, a child, an adolescent, or an adult). Preferably, the “subject,” “host,” or “patient” is a human.

In one embodiment, the present invention provides a method for treating or preventing a Flaviviridae viral infection in a host comprising administering to the host a therapeutically effective amount of at least one compound according to the invention described herein.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

In one embodiment, the methods of the invention are directed for treatment of HCV genotype 1 infection. In another embodiment, the HCV is genotype 1a or genotype 1b.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In treating or preventing one or more conditions/diseases described above, the compounds described above can be formulated in pharmaceutically acceptable formulations that optionally further comprise a pharmaceutically acceptable carrier, adjuvant or vehicle.

In one embodiment, the present invention provides a pharmaceutical composition comprising at least one compound according to the invention described herein and at least one pharmaceutically acceptable carrier, adjuvant, or vehicle, which includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the compounds described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the compounds described above and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds described above and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a compound according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

In one embodiment, the present invention provides a pharmaceutical composition comprising at least one compound according to the invention described herein, and further comprising one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In another embodiment, there is provided a combination therapy of at least one compound according to the invention described herein in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The additional agents for the compositions and combinations include, for example, ribavirin, amantadine, merimepodib, Levovirin, Viramidine, and maxamine

In one combination embodiment, the compound and additional agent are administered sequentially.

In another combination embodiment, the compound and additional agent are administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121A2 (Idenix), WO 02/069903 A2 (Biocryst Pharmaceuticals Inc.), and WO 02/057287 A2 (Merck/Isis) and WO 02/057425 A2 (Merck/lsis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190 (Gilead), and PF-868,554 (Pfizer).

The term “viral NS5A inhibitor” as used herein means an agent that is effective to inhibit the function of the viral NS5A protease in a mammal Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

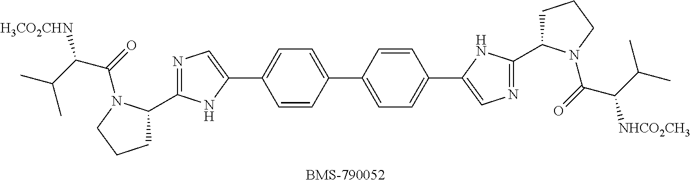

(Gao M. et al. Nature, 465, 96-100 (2010); nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal

“Immunomodulatory agent” as used herein means those agents that are effective to enhance or potentiate the immune system response in a mammal Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class Il interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir(Schering-Plough), VX-950/telaprevir(Vertex) and ITMN-B (InterMune), GS9132 (Gilead), TMC-435350 (Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-lntron™ regimen is 1.0 mg/kg/week subcutaneously for one year. The dose should be administered on the same day of the week.

When administered in combination with ribavirin, the recommended dose of PEG-lntron is 1.5 micrograms/kg/week.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments: viral serine protease inhibitor is HCV serine protease inhibitor; viral polymerase inhibitor is HCV polymerase inhibitor; viral helicase inhibitor is HCV helicase inhibitor.

In one embodiment, the present invention provides a pharmaceutical composition comprising at least one compound according to the invention described herein, one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, and at least one pharmaceutically acceptable carrier or excipient.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein for inhibiting or reducing the activity of viral polymerase in a host.

In a further embodiment, the composition or combination according to the invention further comprises at least one compound according to the invention described herein; one or more additional agents select from non-nucleoside HCV polymerase inhibitors (e.g., HCV-796), nucleoside HCV polymerase inhibitors (e.g., R7128, R1626, R1479), and HCV NS3 protease inhibitors (e.g., VX-950/telaprevir and ITMN-191); and interferon and/or ribavirin.

In one embodiment, the additional agent is interferon α 1A, interferon α 1B, interferon α 2A, or interferon α 2B, and optionally ribavirin.

In one embodiment, the present invention provides a method for treating or preventing a HCV viral infection in a host comprising administering to the host a combined therapeutically effective amounts of at least one compound according to the invention described herein, and one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin.

In one combination embodiment, the compound and additional agent are administered sequentially.

In another combination embodiment, the compound and additional agent are administered simultaneously.

In one embodiment, there is provided a method for inhibiting or reducing the activity of HCV viral polymerase in a host comprising administering to the host a combined therapeutically effective amounts of at least one compound of the invention, and one or more additional agents select from non-nucleoside HCV polymerase inhibitors (e.g., HCV-796) and nucleoside HCV polymerase inhibitors (e.g., R7128, R1626, R1479), interferon and ribavirin.

The combinations referred to above may conveniently be presented for use in the form of a pharmaceutical formulation and thus pharmaceutical formulations or compositions comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of at least one compound of the invention, in combination with the use of one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, for the manufacture of a medicament for treating or preventing a HCV infection in a host.

When the compounds of the invention described herein are used in combination with at least one second therapeutic agent active against the same virus, the dose of each compound may be either the same as or differ from that when the compound is used alone. Appropriate doses will be readily appreciated by those skilled in the art.

The ratio of the amount of a compound according to the invention described herein administered relative to the amount of the additional agent (non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon or ribavirin) will vary dependent on the selection of the compound and additional agent.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### Synthesis of Compounds of the Invention

### Example 1A

### Preparation of Compound 2

Compound 2 can be prepared as depicted below in Scheme A.

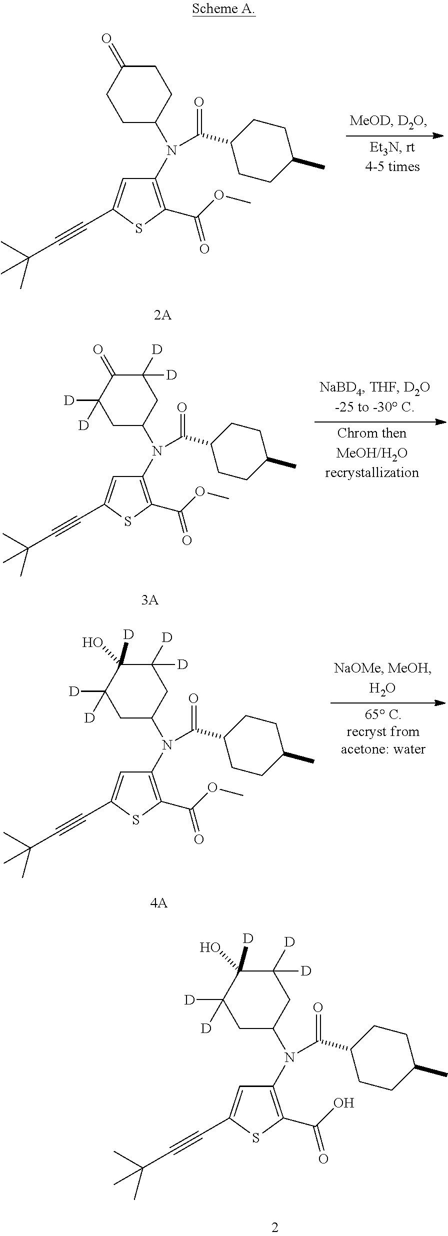

### A. Compound 3A

A solution of starting thiophene (1.44 g, 3.15 mmol) was dissolved in MeOD (99.5% D, 10 mL) and D₂O (99.9% D, 0.5 mL) was added followed by the addition of Et₃N (0.36 mL, d=0.726, 2.37 mmol). The resulting reaction mixture was stirred for 22 h at room temperature then the solvent was removed in vacuo.

The crude material was subjected to the described reaction conditions 4 more times or until there is no detectable des-d₄ material by NMR or LC/MS. The compound was used for the next step without further purification.

### B. Compound 4A

The d₄ thiophene (1.57 g, 3.40 mmol) was dissolved in THF (anhydrous, 15.7 mL) and D₂O (1.57 mL, 99.9% D) was added followed by cooling to −30° C. Sodium borodeuteride (≧98% D, 71.2 mg) was added portionwise over 4 portions (this was weighed out prior to the start of additions). After the addition, the resulting reaction was stirred for 2 h at −25 to −30° C. and analysis showed that the reaction was complete (total consumption of SM by TLC). Aqueous HCl (1M, 2.4 mL) was added and the mixture allowed to warm to rt. Water (75 mL) was added and the resulting mixture extracted with EtOAc (3×75 mL). The organic phases were combined and dried over Na₂SO₄, filtered, and filtrate concentrated in vacuo to provide a crude material which was purified by prep column chromatograph (SP1) with gradient elution 20-100% EtOAc/hexanes. The purified fractions were combined and recrystallized from 2 vol of MeOH to afford a white solid, which was dried to give 1.13 g recryst, 71%.

### C. Compound 2

The purified alcohol (506 mg, 1.09 mmol) was dissolved in 18 mL of a 3:2:1 mixture of THF: MeOH:H₂O and treated with LiOH H₂O (183 mg, 4.36 mmol). The resulting mixture was then warmed to 50° C. for 1.5 hrs. TLC showed that the starting material was totally consumed. The mixture was then concentrated under reduced pressure to remove the solvent. The resulting water phase was made acidic with 4.5 mL of 1M HCl and diluted with water (50 mL). The aqueous phase was extracted with EtOAc (3×50 mL). The EtOAc phases were combined and dried (Na₂SO₄), filtered and the filtrate concentrated in vacuo to provide crude material that was purified by chromatography (SP1, gradient elution using 5-30% MeOH/EtOAc). The final compound contained SiO₂ that was purged by dissolving the compound in EtOAc and filtering off the SiO₂ and evaporating the solvent to give a white solid 385 mg, 78% yield.

### Example 1B

### Alternative Preparation of Compound 2

Alternatively, Compound 2 can be prepared as depicted below in Scheme B.

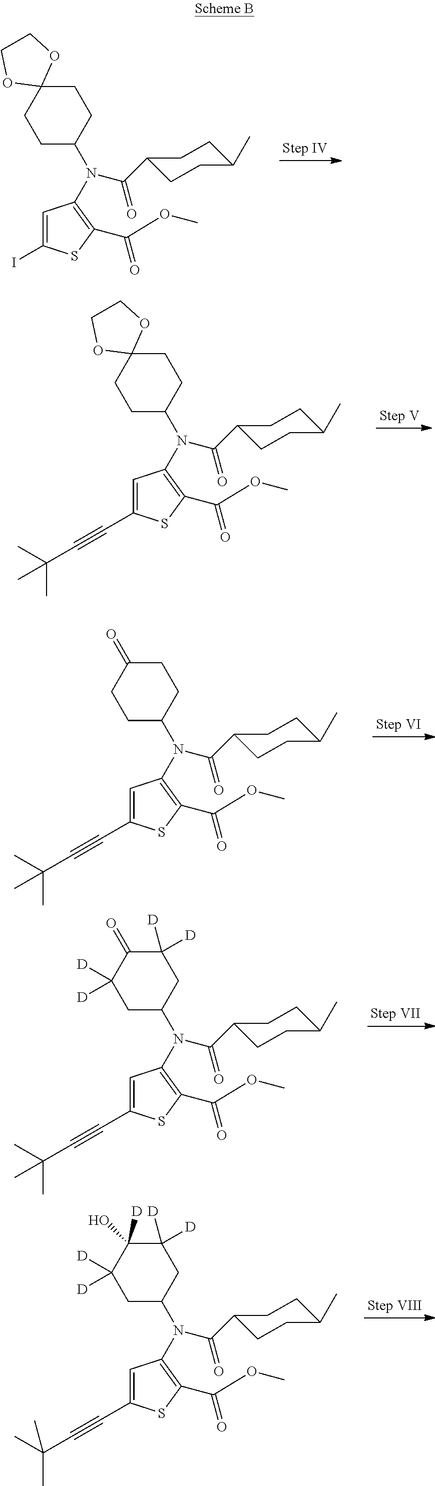

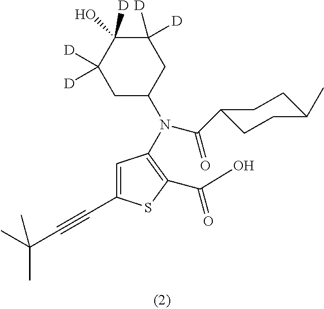

### Step I

A suspension of 3-amino-thiophene-2-carboxylic acid methyl ester (5.0 g, 31.85 mmol) in dry THF (9 mL) is treated with 1,4-cyclohexanedione monoethylene ketal (5.0 g, 32.05 mmol), followed by dibutyltin dichloride (482 mg, 1.59 mmol). After 5 min, phenyl silane (4.3 mL, 34.96 mmol) is added and the reaction mixture is stirred overnight at room temperature. After concentration, the residue is dissolved in EtOAc and washed with NaHCO₃ followed by brine. The organic layer is separated, dried (Na₂SO₄), filtered and concentrated. The residue is purified by column chromatography using 30% ethyl acetate in hexane as eluent to give 3-(1,4-dioxa-spiro[4.5]dec-8-ylamino)-thiophene-2-carboxylic acid methyl ester (4.5 g, 47% yield).

### Step II

### A—Preparation of Trans-4-Methylcyclohexyl Carboxylic Acid Chloride:

Oxalyl chloride (2M in dichloromethane, 17 mL) is added dropwise to a suspension of trans-4-methylcyclohexyl carboxylic acid (2.3 g, 16.2 mmol) in dichloromethane (5 mL) and DMF (0.1 mL). The reaction mixture is stirred for 3 h at room temperature. The volatiles are removed under reduced pressure to obtain the crude acid chloride which is used directly for the next reaction.

B—trans-4-Methylcyclohexyl carboxylic acid chloride is added to a solution of 3-(1,4-dioxa-spiro[4.5]dec-8-ylamino)-thiophene-2-carboxylic acid methyl ester (2.4 g, 8.08 mmol) in toluene (18 mL) followed by pyridine (0.7 mL). The resulting mixture is then stirred for 16 h at reflux. The reaction mixture is diluted with toluene (7 mL) and cooled to 5 C. After the addition of pyridine (1.5 mL) and MeOH (0.8 mL), the mixture is stirred 2 h at 5° C. The white solid is filtered and washed with toluene. The filtrate is washed with 10% citric acid, aq. NaHCO₃, dried (Na₂SO₄) and concentrated. The solid is purified by silica gel column chromatography using 20% EtOAc:hexane as eluent to obtain 3-[(1,4-dioxa-spiro[4.5]dec-8-yl)-(trans-4-methyl-cyclohexanec arb onyl)-amino]-thiophene-2-carboxylic acid methyl ester (2.3 g, 68%).

### Step III

n-BuLi (2 eq.) is added dropwise for 10 min to a cold (−40° C.) solution of diisopropylamine (1 eq.) in dry THF. The reaction mixture is stirred at the same temperature for 30 min. Then a solution of 3-[(1,4-dioxa-spiro[4.5]dec-8-yl)-(trans-4-methyl-cyclohexane-carbonyl)-amino]-thiophene-2-carboxylic acid methyl ester (1 eq.) in THF is added dropwise (35 min) keeping the internal temperature around −40° C. The reaction mixture is stirred for 30 min and a solution of iodine (2 eq.) in THF is added dropwise, stirred for 30 min at the same temperature before being added a sat. solution of NH₄Cl. The reaction mixture is diluted with ethyl acetate and water. The organic layer is separated and washed with 5% sodium thiosulfate solution. The organic layer is separated, dried (Na₂SO₄) and evaporated to a suspension and then added heptane. The suspension is stirred at 0° C. for 30 min, filtered and washed with heptane to obtain 3-[(1,4-dioxa-spiro[4.5]dec-8-yl)-(trans-4-methyl-cyclo-hexanecarbonyl)-amino]-5-iodo-thiophene-2-carboxylic acid methyl ester.

### Step IV

To a 25 mL round bottom flask under nitrogen, 3-[(1,4-dioxa-spiro[4.5]dec-8-yl)-(trans-4-methyl-cyclohexanecarbonyl)-amino]-5-iodo-thiophene-2 carboxylic acid methyl ester (1 eq.), copper iodide (0.025 eq.) and tris(dibenzylidene-acetone) dipalladium (0) (0.01 eq.) are taken. DMF, triethylamine (2.5 eq.) and 3,3-dimethyl-but-1-yne (2 eq.) are added and the reaction mixture is stirred at 40° C. for 2 h under N₂ atmosphere. The reaction mixture is filtered on celite and washed with ethyl acetate. The solution is diluted with water and extracted 2 times with ethyl acetate. The organic phases are combined and washed 3 times with water. The organic layer is separated, dried (Na₂SO₄), evaporated to about 2 mL and then heptane (8 mL) is added. It is evaporated to 2-4 mL and cooled in an ice bath. The resultant white solid is filtered, washed with heptane and dried in oven to obtain 5-(3,3-dimethyl-but-1-ynyl)-3-[(1,4-dioxa-spiro[4.5]dec-8-yl)-(trans-4-methyl-cyc lohexanecarbonyl)-amino]-thiophene-2-carboxylic acid methyl ester.

### Step V

5-(3,3-Dimethyl-but-1-ynyl)-3-[(1,4-dioxa-spiro[4.5]dec-8-yl)-(trans-4-methyl-cyclohexanecarbonyl)-amino]-thiophene-2-carboxylic acid methyl ester (1 eq.) is dissolved in tetrahydrofuran and treated with 3.6 N HCl solution. The reaction is stirred at 40° C. for 5 h. Water is then added and the reaction mixture is cooled to room temperature. The reaction mixture is extracted with ethyl acetate (2×50 mL). The combined extracts are washed with aqueous saturated NaHCO₃ (25 mL) and water (2×50 mL). The organic layer is concentrated to a thick oil and heptane (50 mL) is added to the mixture to precipitate the desired compound which is filtered to give 5-(3,3-dimethyl-but-1-ynyl)-3-[(trans-4-methyl-cyclohexanecarbonyl)-(4-oxo-cyclohexyl)-amino]-thiophene-2-carboxylic acid methyl ester.

### Step VI

To a solution of 5-(3,3-dimethyl-but-1-ynyl)-3-[(trans-4-methyl-cyclohexanecarbonyl)-(4-oxo-cyclohexyl)-amino]-thiophene-2-carboxylic acid methyl ester (1.442 g, 3.150 mmol) in methanol-d4 (10 mL, 99.5% D), D₂O (0.5 ml, 99.9% D) is added followed by Et₃N (0.33 mL, 2.368 mmol). The reaction mixture is stirred at 22 h at room temperature. The solvent is evaporated and the same procedure is repeated for 4 times. The compound, 5-(3,3-dimethyl-but-1-ynyl)-3-[(trans-4-methyl-cyclohexanecarbonyl)-(4-oxo-3,3,5,5-d₄-cyclohexyl)-amino]-thiophene-2-carboxylic acid methyl ester (1.35 g) is used for the next reaction without any further purification.

### Step VII

5-(3,3-Dimethyl-but-1-ynyl)-3-[trans-4-methyl-cyclohexanecarbonyl)-(4-oxo-3,3,5,5-d₄-cyclohexyl)-amino]-thiophene-2-carboxylic acid methyl ester (1.35 g, 2.92 mmol) is dissolved in methanol-d (20 mL) and cooled to 0° C. NaBD₄ (123 mg, 2.92 mmol) is added in one portion, stirred for 1 h at 0° C. and 10% HCl (3 mL) is added. The solvent is removed and diluted with dichloromethane (75 mL). Water (75 ml) is added and then extracted. The organic phase is separated, dried (Na₂SO₄), and evaporated. The residue is purified by column chromatography using ethyl acetate and hexane (20% to 100% ethyl acetate) to give 5-(3,3-dimethyl-but-1-ynyl)-3-[(trans-4-hydroxy-3,3,4,5,5-d5-cyclohexyl)-(trans-4-methyl-cyclohexanecarbonyl)-amino]-thiophene-2-carboxylic acid methyl ester. The compound is further purified by recrystallization in methanol to obtain the pure product (829 mg, 61%).

### Step VIII

5-(3,3-Dimethyl-but-1-ynyl)-3-[(trans-4-hydroxy-3,3,4,5,5-d5-cyclohexyl)-(trans-4-methyl-cyclohexanecarbonyl)-amino]-thiophene-2-carboxylic acid methyl ester (506 mg, 1.09 mmol) is dissolved in a 3:2:1 mixture of THF: methanol: H₂O (18 ml) and then added LiOH.H₂O (183 mg, 4.356 mmol). After 2 h of stirring at 60° C., the reaction mixture is evaporated to dryness. It is neutralized with 1N HCl (4.5 ml). A portion of water (50 ml) and ethyl acetate (50 ml) are added. The organic layer is separated, dried (Na₂SO₄), and concentrated. The residue is purified by column chromatography using methanol and ethyl acetate (5% to 30%) to obtain 5-(3,3-dimethyl-but-1-ynyl)-3-[(trans-4-hydroxy-3,3,4,5,5-d5-cyclohexyl)-(trans-4-methyl-cyclohexane-carbonyl)-amino]-thiophene-2-carboxylic acid (385 mg, 78%).

¹H NMR (400 MHz, dmso): 7.01 (s, 1H), 4.43 (bs, 1H), 4.23 (tt, 1H)), 1.87 (t, 1H), 1.63-1.14 (m, 19H), 0.76-0.75 (m, 4H), 0.56 (td, 2H).

MS found (electrospray): (M+H): 451.39

### Example 1C

### Preparation of Compound 1: 3-[(trans-4-Hydroxy-cyclohexyl)-(trans-4-methylcyclohexanecarbonyl)-amino]-5-d5-phenyl-thiophene-2-carboxylic acid

Compound 1 can be prepared as described in Scheme C below:

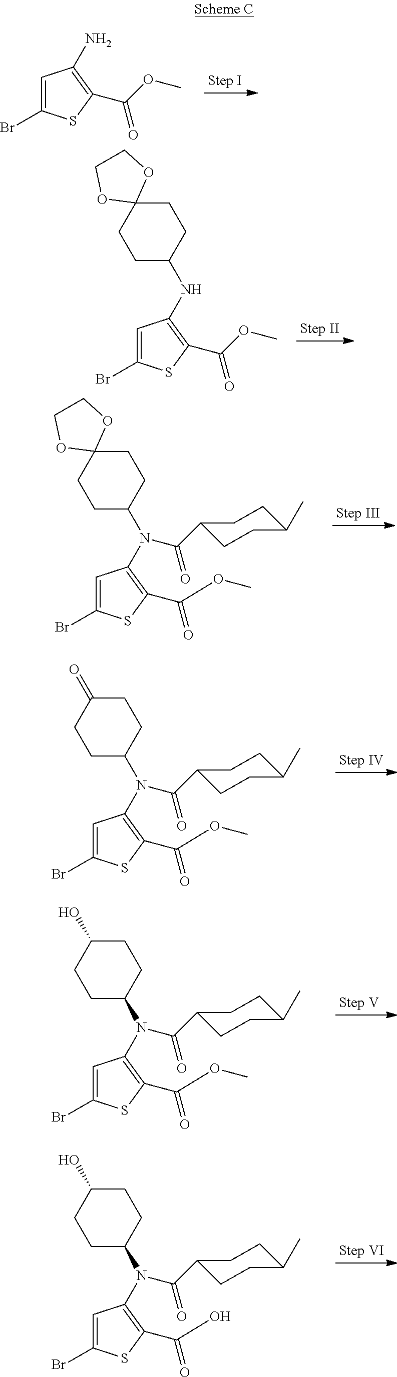

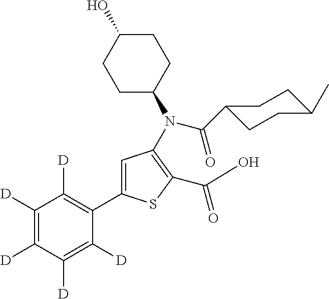 [SMILES: [2H]c1c([2H])c([2H])c(-c2cc(N(C(=O)C3CCC(C)CC3)[C@H]3CC[C@H](O)CC3)c(C(=O)O)s2)c([2H])c1[2H]]

### Step I

A suspension of 3-amino-5-bromo-thiophene-2-carboxylic acid methyl ester (17.0 g, 72.0 mmol) in dry THF (21 mL) is treated with 1,4-cyclohexanedione monoethylene ketal (11.3 mg, 72.0 mmol), followed by dibutyltin dichloride (1.098 g, 3.6 mmol). After 5 min, phenyl silane (9.74 mL, 79.2 mmol) is added and the reaction mixture is stirred overnight at room temperature. After concentration, the residue is dissolved in EtOAc washed with NaHCO₃ then brine. The organic layer is separated, dried on Na₂SO₄, filtered and concentrated. The crude material is diluted with hexane (500 mL). After filtration, the mother liquor is evaporated to dryness to give 5-bromo-3-(1,4-dioxa-spiro[4.5]dec-8-ylamino)-thiophene-2-carboxylic acid methyl ester (24.79 g, 92% yield). ¹H NMR (CDCl₃, 400 MHz): 6.90 (br s, 1H), 6.65 (s, 1H), 3.95 (s, 4H), 3.78 (s, 3H), 3.35 (m, 1H), 2.00 (m, 2H), 1.80 (m, 2H), 1.65 (m, 4H).

### Step II

### A—Preparation of Trans-4-Methylcyclohexyl Carboxylic Acid Chloride

Oxalyl chloride (2M in DCM, 117 mL) is added dropwise to a suspension of trans-4-methylcyclohexyl carboxylic acid (16.6 g, 117 mmol) in DCM (33 ml) and DMF (0.1 mL) the reaction mixture is stirred 3 h at room temperature. DCM is removed under reduced pressure and the residue is co-evaporated with DCM. The residue is dissolved in toluene to make a 1M solution.

### B—Preparation of the Target Compound

The 1M solution of trans-4-methylcyclohexyl carboxylic acid chloride is added to a solution of 5-bromo-3-(1,4-dioxa-spiro[4.5]dec-8-ylamino)-thiophene-2-carboxylic acid methyl ester (24.79 g, 65 mmol) in toluene (25 mL) followed by pyridine (5.78 mL, 71.5 mmol). The resulting mixture is then stirred for 16 h at reflux. The reaction mixture is diluted with toluene (60 mL) and cooled down to 5° C. After the addition of pyridine (12 mL) and MeOH (5.6 mL), the mixture is stirred 2 h at 5° C. The white suspension is filtered off and the toluene is added to the mother liquor. The organic phase is washed with 10% citric acid, aq. Sat NaHCO₃, dried (Na₂SO₄) and concentrated. The residue is triturated in boiling hexane (1500 mL). The reaction mixture is allowed to cool down to room temperature. The reaction flask is immersed into ice bath, and stirred for 30 min; white solid is filtered off, and washed with cold hexane (225 mL). The solid is purified by silica gel column chromatography using 20% EtOAc:hexanes as eluent to obtain 5-bromo-3-[(1,4-dioxa-spiro[4.5]dec-8-yl)-(trans-4-methyl-cyclohexanecarbonyl)-amino]-thiophene-2-carboxylic acid methyl ester (10.5 g, 32%). ¹H NMR (CDCl₃, 400 MHz): 6.84 (s, 1H), 4.62 (m, 1H), 3.90-3.82 (m, 4H), 3.80 (s, 3H), 1.92-1.81 (m, 2H), 1.77-1.11 (m, 14H), 1.79 (d, 3H), 0.77-0.59 (m, 2H).

### Step III

The 5-bromo-3-[(1,4-dioxa-spiro[4.5]dec-8-yl)-(trans-4-methylcyclohexane-carbonyl)-amino]-thiophene-2-carboxylic acid methyl ester (8.6 g, 17 mmol) is dissolved in tetrahydrofuran (100 mL) and treated with 3N HCl solution (50 mL). The reaction is stirred at 40° C. for 3 h. The reaction mixture is evaporated under reduced pressure. The residue is dissolved in EtOAc and washed with aq. sat. NaHCO₃ solution. The organic layer is separated, dried on Na₂SO₄, filtered and concentrated to give 5-bromo-3-[(trans-4-methyl-cyclohexanecarbonyl)-(4-oxo-cyclohexyl)-amino]-thiophene-2-carboxylic acid methyl ester as a solid (7.4 g, 95%).

### Step IV

To a cold (0° C.) solution of 5-bromo-3-[(trans-4-methyl-cyclohexanecarbonyl)-(4-oxo-cyclohexyl)-amino]-thiophene-2-carboxylic acid methyl ester (5.9 g, 12.9 mmol) in MeOH (50 mL) under N₂ is added NaBH₄ (250 mg, 6.4 mmol, 0.5 eq.) portion wise (approx. 30 minutes). After the addition is completed, checked for reaction completion by TLC Hexane:EtOAc (1:1). HC12% (10 mL) is added and stirred for 15 min. The reaction mixture is concentrated under vacuum to dryness. The reaction mixture is recuperated with water (25 mL) and extracted with EtOAC. The organic phases are combined and dried over MgSO₄ and concentrated to dryness. The residue is purified by silica gel column chromatography using EtOAc:hexanes as eluent to obtain 5-bromo-3-[(trans-4-hydroxy-cyclohexyl)-(trans-4-methyl-cyclohexane-carbonyl)-amino]-thiophene-2-carboxylic acid methyl ester (4.5 g, 77% yield) as a solid.

### Step V

5-Bromo-3-[(trans-4-hydroxy-cyclohexyl)-(trans-4-methyl-cyclohexane-carbonyl)-amino]-thiophene-2-carboxylic acid methyl ester (3.0 g, 6.68 mmol) is dissolved in a 3:2:1 mixture of THF: methanol: H₂O (50 mL) and treated with a 1N solution of LiOH.H₂O (8.0 mL, 8.0 mmol). After 2 h of stirring at 60° C., the reaction mixture is evaporated to dryness and used as it is for the next step.

### Step VI

A solution of 5-bromo-3-[(trans-4-hydroxy-cyclohexyl)-(trans-4-methyl-cyclohexanecarbonyl)-amino]-thiophene-2-carboxylic acid (509 mg, 1.145 mmol) and d5-phenylboronic acid (150 mg, 1.179 mmol) in a mixture of DME (10 mL) and 2M aqueous Na₂CO₃ (5 mL) is treated with Pd(PPh₃)₄ (66.2 mg, 0.0576 mmol). The reaction mixture is heated at reflux for 30 min. The reaction mixture is diluted with ethyl acetate and water. The organic layer is separated and dried (Na₂SO₄) and concentrated. The residue is purified with silica gel column chromatography using CH₂Cl₂:MeOH as eluent to provide 3-[trans-4-hydroxy-cyclohexyl)-(trans-4-methylcyclohexanecarbonyl)-amino]-5-d5-phenyl-thiophene-2-carboxylic acid (278 mg, 54%): ¹H (400 MHz, CDCl₃): δ 7.02 (s, 1H), 4.59 (t, 1H), 3.47 (m, 1H), 2.22-1.05 (m, 16H), 0.90-0.49 (m, 5H); MS found (electrospray): (M−H): 447.34

### Example 1D

### Preparation of Compound 5

Compound 5 can be prepared as depicted below in Scheme D.

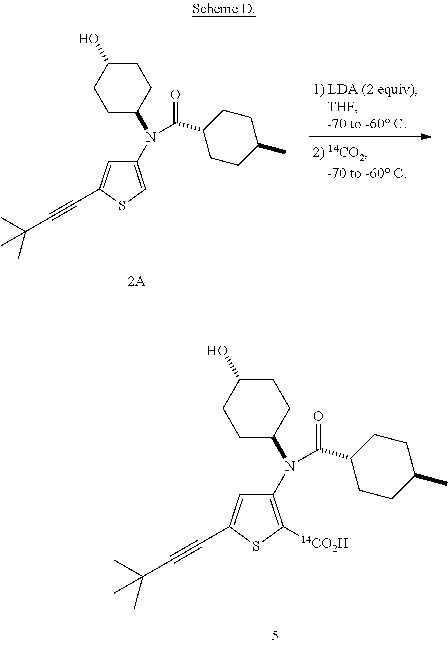

Compound 2A was prepared by decarboxylation of the corresponding carboxylic acid using copper powder in quinoline at 200° C. The starting carboxylic acid (20.0 g, 44.9 mmol) was added to a 500 mL roundbottom flask fitted with a condenser, N₂ inlet, and thermocouple. Copper powder (3.51 g, 55.3 mmol) was added along with quinoline (200 mL). The mixture was then heated to 200° under nitrogen for 6 h. After cooling to RT, the mixture was dissolved in EtOAc (700 mL) and filtered through a pad of silica gel then rinsed with EtOAc (600 mL). The filtrate was washed with 2N HCl (3×350 mL), then water (1×350 mL), and brine (1×350 mL). The organic phase was dried over Na₂SO₄, filtered, and concentrated in vacuo to a orange-tan solid. The crude solid was then suspended in heptane (100 mL) and stirred for 48 h). After filtering, the solid was crystallized from hot EtOAc (50 mL) to obtain 11.8 g of desired product.

Two equivalents of LDA was prepared by the addition of n-BuLi (2.5 M in hexane, 0.75 mmol) to a diisopropyl amine (1 g, 10 mmol) solution in THF (10 mL) at −70° C. The solution is typically warmed up to nearly 0° C. for 15 minutes then cooled back down to −70° C. and stirred for about 1 h for the addition of compound 2A. Compound 2A (1 g, 2.49 mmol) was dissolved in THF (20 mL) and added to a mixture over 15 min while keeping the temperature below −60° C. This mixture was stirred for 90 min at −70 to −60° C. Then CO₂ (g) was introduced at a moderate rate over 40 min while keeping the reaction mixture below −60° C. The reaction was monitored by HPLC and stopped after the reaction mixture was >95% complete. CO₂ addition was stopped and the mixture poured very slowly into a solution of 5 mL of water with 20 mL of THF at 0° C. Caution: CO₂ really rips out at the higher temperature. One may want to do this in a more controlled fashion. The material was allowed to warm back up to 0° C. and acidified with HCl (30 mL, 1N, 30 mmol) added keeping the reaction mixture at about 0° C. The mixture was stirred at 0° C. for 30 min, diluted with EtOAc (50 mL). Phases were separated and the water phase extracted a 2nd time with EtOAc (50 mL). The combined organic phase was washed with brine (2×20 mL), dried over Na₂SO₄, filtered and concentrated in vacuo. The crude solid proved >95% purity and was stirred in heptane for 2 h filtered and dried in vacuo to give 800 mg of compound 5 (cold) in 98% purity. ¹H NMR (500 MHz, DMSO-d₆) 0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### Example 1E

### Preparation of Compound 4

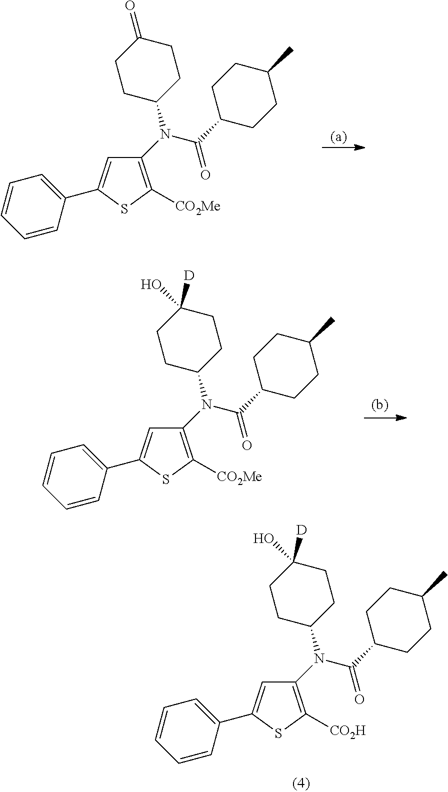

### Step (a)

Methyl 3-[(4-trans-methylcyclohexanecarbonyl)-(4-oxocyclohexyl)amino]-5-phenyl-thiophene-2-carboxylate (1 g, 2.2 mmol) was taken in THF (57 mL) and four drops of D₂O and the reaction mixture cooled to −25° C. Then added sodium borodeuteride (92 mg, 2.2 mmol). Stirred for 4 hours, then the reaction was quenched with 1N HCl, (25 mL) then the reaction mixture diluted with ethyl acetate and water. The organic layer was washed with brine and dried over Na₂SO₄. The solution was concentrated and the product purified by silica gel chromatography on an ISCO 40 “Gold” column eluted with a gradient of 0-100% EtOAc in hexane to afford methyl 5-(phenyl)-3-[(4-trans-hydroxy-4-deuterocyclohexyl)-(4-trans-methylcyclohexanecarbonyl)amino]thiophene-2-carboxylic acid

Yield 400 mg: LCMS [M+H] 457.5; Rt=1.41 min

### Step (b)

A solution of methyl 5-(phenyl)-3-[(4-trans-hydroxy-4-deuterocyclohexyl)-(4-trans-methylcyclohexanecarbonyl)amino]thiophene-2-carboxylic acid (400 mg, 0.88 mmol) in MeOH (20 mL) was cooled to 10° C. 2M NaOH (10 mL, 20 mmol) was added and the reaction stirred at RT for 18 h. MeOH was evaporated and the mixture treated with EtOAc and 1N HCl, then extracted (2×). Removal of solvent afforded a glass, which was triturated with MeCN, filtered, washed with acetonitrile and dried: LCMS [M+H] 443.0; Rt=0.91 min; NMR (300 MHz, CDCl₃) dH 1H 7.67 (m, 2H), 7.44 (m, 3H), 7.06 (s, 1H), 5.92 (br 1H), 4.62 (t, 1H), 2.13-1.91 (m, 5H), 1.80-1.00 (m, 11H), 1.30 (s, 9H), 0.79 (d, J=9.4 Hz, 3H), 0.75-0.50 (m, 2H).

### Example 1F

### Preparation of Compound 3

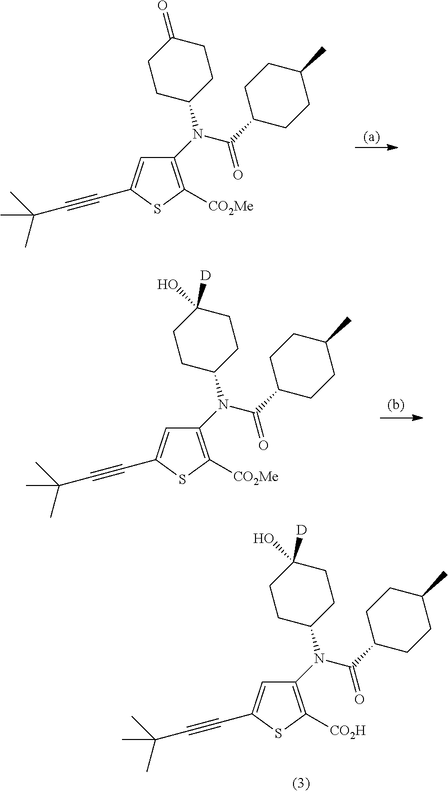

### Step (a)

Methyl 3-[(4-trans-methylcyclohexanecarbonyl)-(4-oxocyclohexyl)amino]-5-(3,3-dimethylbut-1-yn-1-yl)-thiophene-2-carboxate (350 mg, 0.76 mmol) was taken in THF (20 mL) and two drops of water, and the reaction mixture cooled to −25° C. Then added sodium borodeuteride (NaBD₄: 32 mg, 0.76 mmol) and the reaction stirred for 4 h. The reaction was quenched with 1N HCl, then the reaction mixture diluted with ethylacetate and water. Extracted the organic layer, washed with brine and dried over Na₂SO₄. Concentrated then was purified by silica gel chromatography to afford methyl 5-(3,3-dimethylbut-1-yn-1-yl)-3-[(4-trans-hydroxy-4-deuterocyclohexyl)-(4-trans-methylcyclohexanecarbonyl)amino]thiophene-2-carboxylic acid

Yield 200 mg: LCMS [M+H] 461.4; Rt=5.8 min

### Step (b)

Methyl 5-(3,3-dimethylbut-1-yn-1-yl)-3-[(4-trans-hydroxy-4-deuterocyclohexyl)-(4-trans-methylcyclohexanecarbonyl)amino]thiophene-2-carboxylic acid (200 mg, 0.43 mmol) was taken in THF (20 mL) and H2O 2O (4 mL) followed by addition of LiOH (10.4 mg, 0.43 mmol). The reaction mixture was stirred at RT overnight. The reaction mixture was washed with ethyl acetate, then the aqueous layer was acidified with 1N HCl. and extracted with ethyl acetate. The extracts were washed with brine and dried over Na2SO4. The solution was concentrated to give a yellow oil, which was purified by slica gel chromatography and C18 reversed phase HPLC (gradient of 60-95% methanol in H2O 2O) to give desired product (compound 3). Yield 194 mg: LCMS [M+H] 447.5; Rt=5.42 min; NMR (300 MHz, d6-DMSO) dH 1H 13.44 (s, 1H), 7.16 (s, 1H), 4.44 (s, 1H), 4.27 (t, J=10.0 Hz, 1H), 1.90-1.69 (m, 4H), 1.57 (d, J=11.4 Hz, 5H), 1.30 (s, 9H), 1.19 (d, J=9.4 Hz, 3H), 0.91-0.78 (m, 2H), 0.76 (d, J=6.5 Hz, 3H), 0.67-0.37 (m, 2H).

### Example 2

### HCV Replicon Assay

### A. Principle

This procedure below describes the HCV replicon assay using a Huh7 hepatoma cell line harboring a highly cell culture-adapted replicon (genotype 1b) (hereafter named cell line ET). The ET cells contained the highly cell culture-adapted replicon I₃₈₉luc-ubi-neo/NS3-3′/5.1 construct that carried, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Viral. 2001, 75, 4614-4624). A replicon cell line W11.8, containing the 1a genotype of HCV was also used. These two cell lines (genotype 1b and 1a) allowed measurement of RNA replication and translation by measuring luciferase activity (against genotype 1b) or by measuring the NS5A level using the ELISA assay (against genotype 1a). It was shown that the luciferase activity tightly followed the replicon RNA level in the ET cells. ET cell lines were maintained in cultures at a sub-confluent level (<85%). The culture media used for cell passaging consisted of DMEM (Gibco BRL Laboratories, Mississauga, ON, Canada) supplemented with 10% fetal bovine serum with 1% penicilin/streptomycin, 1% glutamine, 1% sodium pyruvate, 1% non-essential amino acids, and 180 μg/ml of G418 final concentration.

### B. Measurement of Luciferase Activity (Luci-ET-1b)

For the treatment of the cells with the testing drug, the culture medium was removed from the 175 cm² T-flask by aspiration. Cell monolayer was rinsed with 10 mL of PBS 1× at room temperature. PBS was removed by aspiration. Cells were trypsinized using 1 mL of Trypsin/EDTA. Flask were incubated at 37° C. (incubator) for 7 minutes. Complete medium (9 mL) with no G418 and no phenol red was then added. Cell clumps were disrupted by pipetting up and down several times. The cell suspension was then transferred to a 50 mL Falcon polypropylene tube. Cells were then counted several times using the hemacytometer. Cells were diluted at 30 000 cells/mL with complete DMEM with no G418 and no phenol red, then transferred into a sterile reservoir. Using a multichannel pipet, approximately 3000 viable cells (100 μL) were plated per well in a white opaque 96-well microtiter plate. After an incubation period of 2-4 hours at 37° C. in a 5% CO₂ incubator, compounds were added at various concentrations.

Compounds under testing were resuspended in DMSO at a stock concentration of 100 mM. Then, they were diluted at twice the final concentration in the same medium (without G418) described earlier, in sterile 96-deep well plate and according to a particular template. One volume (100 μL) of each compound dilution was then added to each well that contains cells or in control wells with no cells. Final drug concentrations were usually between 200 μM and 0.0001 μM. Ten wells were used as positive control without drug. Cells were further incubated for 4 days at 37° C. in a 5% CO₂ incubator. A control compound was used as an internal standard at the same concentrations described above.

Following the incubation time of four days, the culture media was removed and quickly dried upside down on a stack of sterile absorbing papers. Cells were then lysed by the addition of 95 μL of the luciferase buffer A using a mutichannel pipet, sealed using TopSeal™ adhesive sealing film and the reaction mixture was incubated at room temperature and protected from direct light for at least 10 minutes. Plates were read for luciferase counts using a luminometer (Wallac MicroBeta Trilux, Perkin Elmer™, MA, USA).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC₅₀ values are summarized in Table 1:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### C. Elisa Assay (ELISA W 11.8-1a)

Replicon cell lines W11.8 containing a sub-genomic replicon of genotype la was used for the HCV Replicon Cell-Based detection using the ELISA. The RNA replication in presence of different drug concentrations was indirectly measured in these cell lines by the level of NS5A protein content upon drug treatment for four days. The NS5A is a non-structural protein of HCV and is used as marker of HCV replication in this assay.

For the treatment of the cells with the testing drug, Culture medium was removed from the 175 cm² T-flask by aspiration. Cell monolayer was rinsed with 10-20 mL of PBS 1× at room temperature. PBS was removed by aspiration. Cells were trypsinized using 3 mL of Trypsin (0.25%)/EDTA (0.1%) solution. Flasks were incubated at 37° C. (incubator) for 7 minutes. Complete medium (9 mL) without G418 is then added. Cell clumps were disrupted by pipetting up and down several times.

The cell suspension was then transferred to a 50 mL Falcon polypropylene tube. Cells were then counted several times using the haemocytometer. Cells were diluted at 50,000 cells/mL with complete DMEM without G418, then transferred into a sterile reservoir. Using a multichannel pipet, approximately 5,000 viable cells (100 μL) were plated per well in a white opaque 96-well microtiter plate. After an incubation period of 2-4 hours at 37° C. in a 5% CO₂ incubator, compounds were added at various concentrations.

Drugs were resuspended in DMSO at a stock concentration of 100 mM or 10 mM. In some cases (drugs with a potency below nmolar values), it was necessary to dilute compounds in DMSO at 1 mM or 100 μM as a starting solution. Then, drugs were diluted at twice the final concentration in the same medium (without G418) described earlier, in sterile 96-deep well plate and according to a particular template (see Appendix). One volume (100 μL) of each drug dilution was then added to each well that contains cells.

Sixteen wells were used as control (0% inhibition) without drug. Eight wells were used as background control (100% inhibition) containing 2 μM (final concentration) of the reference compound. The reference compound at 2 μM was shown to inhibit the NS5A expression at ≈100% and is nontoxic to the cells. Values from 100% inhibited wells were averaged and used as the background value. Cells are further incubated for 4 days at 37° C. in a 5% CO₂ incubator.

For the measurement of NS5A protein content, following the incubation time of four days, the media was throwed into an appropriate waste container by inverting the plate. Any residual liquid was removed by tapping gently on absorbent paper several times. The plates were then washed once with 150 μL of PBS per well, and then incubated for 5 minutes at room temperature on a shaker (500 rpm). 150 μL per well of cold (−20° C.) fixative solution (50% methanol/50% acetone mix) was added into the plates, and the plates was incubated for 5 minutes at room temperature. The pleates were then inverted, and any residual liquid was removed by tapping gently on absorbent paper several times. The plates were then washed twice with 150 μL of PBS per well, and incubated for 5 minutes at room temperature on a shaker (500 rpm) for each wash. 150 μL of blocking solution per well was added into the plates. The plates were then sealed using TopSeal™ adhesive sealing films and incubated for one hour at 37° C. or at 4° C. overnight to block non-specific sites.

The plates were inverted and the blocking solution was dumped into an appropriate waste container. Any residual liquid was removed by tapping gently on absorbent paper several times. The plates were then washed twice with 150 μL of PBS per well and once with 150 μL of PBSTS solution per well, and then incubated for 5 minutes at room temperature on a shaker (500 rpm) for each wash. Then, was add into the plates 50 μL per well of anti-human NS5A antibody (Ab1) diluted 1/1,000 in the blocking solution. The plates were then sealed using TopSeal™ adhesive sealing films and incubate at 4° C. overnight.

Next day, the plates were invered to dump solution into an appropriate waste container. The plates then wwere gently tapped on absorbent paper several times to remove residual liquid. The plates were washed five times with 150 μL of PBS per well, and incubated for 5 minutes at room temperature on a shaker (500 rpm) for each wash. Then was add into the plates 50 μL per well of peroxidase-conjugated donkey anti-mouse antibody (Ab2) diluted 1/10,000 in the blocking solution. The plates were then sealed using TopSeal™ adhesive sealing films and incubate at room temperature for 3 hours on a shaker (500 rpm). Towards the end of the 3 hours incubation, the commercially available chemiluminescent substrate solution was prepared. A mixture of equal volumes of the luminol/enhancer and stable peroxide reagents was prepared and protected from light. The plates were then inverted to dump solution into an appropriate waste container. Any residual liquid was removed by tapping gently on absorbent paper several times. The plates were washed four times with 150 μL of PBSTS solution per well and once with 150 μL of PBS, and then incubated for 5 minutes at room temperature on a shaker (500 rpm) for each wash. 100 μL of substrate solution per well was then added into the plates. The plates were then sealed using TopSeal™ adhesive sealing films and incubate for 1 minute at room temperature on a shaker (500 rpm), and then ncubated between 30 and 60 minutes at room temperature (protect from light) prior to reading the luminescence (relative light units) on the Analyst HT plate reader (LJL Default Luminescence Method).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC50 values are summarized in Table 1:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### Example 3

### [³H]Thymidine Incorporation Assay

A total of 2,000 cells/well were seeded in 96-well cluster dishes in a volume of 100 [mu]l of DMEM (Wisent., St Bruno, QC) supplemented with 10% FBS (Wisent., St Bruno, QC) and 2 mM glutamine (Life Technologies, Inc.). Penicillin and streptomycin (Life Technologies, Inc.) are added to 500 U/mL and 50 μg/mL final concentrations, respectively. After an incubation of at least 3 h at 37° C. in an atmosphere of 5% CO₂, compounds, prepared at twice the final concentration, are added to the cells. Eleven serial two to four-fold dilutions of drugs are tested in duplicate plates. After 72-h incubation, a volume of 20 μL of a 10 piCi/mL solution of [3H] methyl thymidine (Amersham Life Science, Inc., Arlington Heights, III; 2 Ci/mmol) in culture medium is added and the plates are incubated for a further a 24 h at 37° C. Cells are then washed with phosphate-buffered saline (PBS), trypsinized for 2 min, and collected onto a fiberglass filter using a Tomtec cell harvester (Tomtec, Orange, Conn.). Filters are dried at 37° C. for 1 h and placed into a bag with 4.5 mL of liquid scintillation cocktail (Wallac Oy, Turku, Finland). The accumulation of [3H] methyl thymidine, representing viable replicating cells, is measured using a liquid scintillation counter (1450-Microbeta; Wallac Oy). Ref SOP: 265-162-03. For this experiment, the cell lines used are; Huh-7 ET (cells derived from the Huh-7 cell line (hepatocellular carcinoma, human) and containing a HCV sub-genomic replicon), Molt-4 (peripheral blood, acute lymphoblastic leukemia, human), DU-145 (prostate carcinoma, metastasis to brain, human), Hep-G2 (hepatocellular carcinoma, human), and SH-SYSY (neuroblastoma, human) cells.

The 50% cytotoxic concentrations (CC₅₀) for cell toxicity were determined from dose response curves using six to eight concentrations per compound in triplicate. Curves were fitted to data points using non-linear regression analysis, and IC₅₀ values were interpolated from the resulting curve using GraphPad Prism software, version 2.0 (GraphPad Software Inc., San Diego, Calif., USA).

CC₅₀ values of compounds of the invention are summaries in Table 1:
A: CC₅₀ value (mean)≧100 μM;
B: 10 μM≦CC₅₀ value (mean)<100 μM;
C: CC₅₀ value (mean)≦10 μM.

TABLE 1
IC50, LCMS and NMR data of the compounds described in FIG. 1

| | | HCV- | | | | |
|---|---|---|---|---|---|---|
| | HCV- | Replicon- | | | | |
| | Replicon- | ELISA-1a- | | LCMS | LCMS | |
| Compounds | 1b- IC50 | IC50 | CC50 | [M + H]⁺ | RT | NMR |
| 1 | B | | B | | | ¹H (400 MHz, |
| | | | | | | CDCl₃): δ 7.02 (s, |
| | | | | | | 1H), 4.59 (t, 1H), |
| | | | | | | 3.47 (m, 1H), 2.22- |
| | | | | | | 1.05 (m, 16H), 0.90- |
| | | | | | | 0.49 (m, 5H) |
| 2 | | | | 451.39 | | ¹H NMR (400 MHz, |
| | | | | | | dmso): 7.01 (s, 1H), |
| | | | | | | 4.43 (bs, 1H), 4.23 |
| | | | | | | (tt, 1H)), 1.87 (t, 1H), |
| | | | | | | 1.63-1.14 (m, 19H), |
| | | | | | | 0.76-0.75 (m, 4H), |
| | | | | | | 0.56 (td, 2H) |
| 3 | A | A | A | 447.49 | 5.42 | 1H NMR (300 MHz, |
| | | | | | | DMSO) d 7.16 (s, |
| | | | | | | 2H), 4.44 (s, 3H), |
| | | | | | | 3.30 (s, 8H), 2.50 |
| | | | | | | (dt, J = 3.5, 1.7 Hz, |
| | | | | | | 12H), 2.07 (s, 1H), |
| | | | | | | 1.96-1.29 (m, 38H), |
| | | | | | | 1.29-1.27 (m, 1H), |
| | | | | | | 0.97 (dd, J = 128.0, |
| | | | | | | 7.9 Hz, 18H), −0.00 |
| | | | | | | (s, 3H). |
| 4 | B | | A | 443 | 0.91 | 1H NMR (300 MHz, |
| | | | | | | CDCl3) d 7.69, 7.68, |
| | | | | | | 7.66, 7.66, 7.51, |
| | | | | | | 7.50, 7.48, 7.46, |
| | | | | | | 7.43, 7.41, 7.28, |
| | | | | | | 7.06, 5.92, 4.62, |
| | | | | | | 4.16, 4.13, 3.24, |
| | | | | | | 2.19, 2.13, 2.07, |
| | | | | | | 2.02, 1.98, 1.95, |
| | | | | | | 1.91, 1.72, 1.67, |
| | | | | | | 1.63, 1.53, 1.50, |
| | | | | | | 1.47, 1.46, 1.43, |
| | | | | | | 1.40, 1.36, 1.30, |
| | | | | | | 1.28, 1.26, 1.25, |
| | | | | | | 1.22, 1.14, 1.10, |
| | | | | | | 1.07, 1.03, 0.80, |
| | | | | | | 0.78, 0.71, 0.66, |
| | | | | | | 0.62. |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-50. (canceled)

51. A compound selected from the structural formulae depicted below: 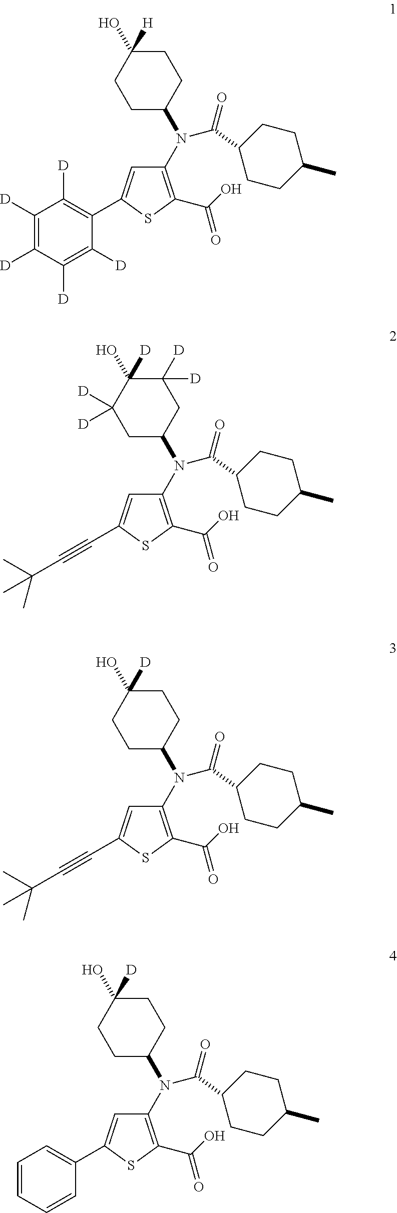 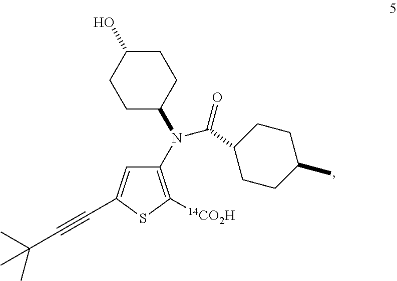
or a pharmaceutically acceptable salt thereof.

52. A pharmaceutical composition, comprising a compound of claim 51, and a pharmaceutically acceptable carrier or excipient.

53. (canceled)

54. A method of inhibiting or reducing the activity of HCV polymerase in a biological in vitro sample, comprising administering to the sample an effective amount of a compound of claim 51.

55. A method of treating a HCV infection in a subject, comprising administering to the subject a therapeutically effective amount of a compound of claim 51.

56. A method of inhibiting or reducing the activity of HCV polymerase in a subject, comprising administering to the subject a therapeutically effective amount of a compound of claim 51.

57. The method of claim 55, further comprising co-administering one or more additional therapeutic agents to the subject.

58. The method of claim 57, wherein the additional therapeutic agents include an anti-HCV drug.

59. The method of claim 58, wherein the anti-HCV drug is an HCV protease inhibitor.

60. The method of claim 59, wherein the HCV protease inhibitor is an HCV NS3 inhibitor.

61. The method of claim 59, wherein the HCV protease inhibitor is VX-950.

62. The method of claim 59, wherein the HCV protease inhibitor is an HCV NS5A inhibitor.

63. The method of claim 58, wherein an interferon and/or ribavirin is co-administered.

64. The method of claim 63, wherein the interferon is a pegylated interferon.

65. (canceled)

66. (canceled)

67. The method of claim 55, wherein the HCV is genotype 1.

68. (canceled)

69. A method of preparing a compound represented by Structural Formula (I): 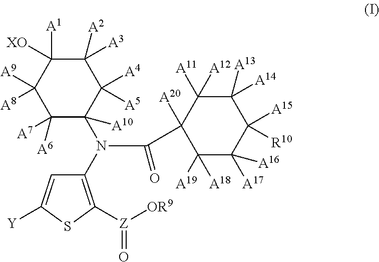
or a pharmaceutically acceptable salt thereof, wherein the variables of Structural Formula (I) are each and independently as described in claim 51, comprising the step of reducing compound (1h) to form compound (1i) that is a compound of Structural Formula (I) wherein X is —H and R⁹ is -Me: 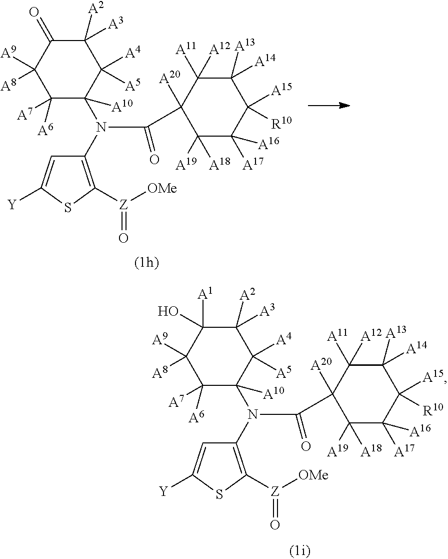
wherein the variables of compounds (1h) and (1i) are each and independently as described in claim 51, and R^{k} is H, C₁₋₆ alkyl or benzyl.

70. The method of claim 69, further comprising the step of hydrolyzing the group represented by —Z(O)OMe of Compound (1i) under a suitable hydrolysis condition to form the corresponding —Z(O)OH.

71. (canceled)
